(19) United States
(12) Patent Application Publication
(10) Pub. No.: US 2011/0034419 A1
(43) Pub. Date: Feb. 10, 2011

(54) Cardioprotective Drugs and Diagnostics for Assessing Risk of Cardiovascular Disease

(75) Inventors: Kelley M. Argraves, Johns Island, SC (US); W. Scott Argraves, Johns Island, SC (US); Alan T. Remaley, Bethesda, MA (US); Amar Sethi, Issaquah, WA (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: MUSC Foundation for Research Development, Charleston, SC (US)

(21) Appl. No.: 12/824,904

(22) Filed: Jun. 28, 2010

Related U.S. Application Data
(60) Provisional application No. 61/221,056, filed on Jun. 27, 2009.

(51) Int. Cl. A61K 31/661; A61K 31/164; A61P 9/10; C12Q 1/02; H01J 49/00

(52) U.S. Cl. 514/114; 514/625; 435/29; 250/281

(57) ABSTRACT

Disclosed are methods of diagnosing cardiovascular disease comprising measuring sphingolipids. Also disclosed are methods of predicting cardiovascular disease comprising measuring sphingolipids. Also disclosed are methods of identifying subjects at risk of developing cardiovascular disease comprising measuring sphingolipids.

### II. CROSS REFERENCE TO RELATED APPLICATIONS

This application claims benefit of U.S. Provisional Application No. 61/221,056, filed Jun. 27, 2009. Application No. 61/221,056, filed Jun. 27, 2009, is hereby incorporated herein by reference in its entirety.

### I. STATEMENT REGARDING FEDERALLY SPONSORED RESEARCH

This invention was made with government support under HL080404 awarded by National Institutes of Health. The government has certain rights in the invention.

### III. BACKGROUND

High density lipoproteins (HDL) are physiological carriers of sphingolipids in human plasma. Findings from a growing number of studies indicate that at least one HDL associated sphingolipid, sphingosine 1-phosphate (S1P), is a mediator of many of the cardioprotective effects of HDL such as HDL-S1P-mediated suppression of inflammatory processes including reduction of monocyte and lymphocyte interaction with the endothelium, reduction in numbers of circulating lymphocytes and decreased pro-inflammatory cytokine secretion. Epidemiological studies demonstrate an inverse correlation between plasma levels of HDL and risk of cardiovascular disease. However, some individuals with high HDL levels still have cardiovascular disease. The failure of high plasma levels of HDL to be cardioprotective in some individuals is a deficiency in HDL-associated molecules, such as sphingolipids.

### IV. SUMMARY OF THE INVENTION

Disclosed herein are methods of diagnosing cardiovascular disease in a subject, comprising the steps of:
a. collecting body fluid from the subject;
b. measuring the level of at least one sphingolipid in the body fluid;
c. diagnosing cardiovascular disease in a subject based on the measured level of sphingolipids.

Also disclosed herein are methods predicting cardiovascular disease in a subject, comprising the steps of:
a. collecting body fluid from the subject;
b. measuring the level of at least one sphingolipid in the body fluid;
c. identifying a subject at risk for cardiovascular disease based on the measured level of sphingolipids.

Also disclosed herein are methods of identifying a subject at risk of developing cardiovascular disease, comprising the steps of:
a. collecting body fluid from the subject;
b. measuring the level of at least one sphingolipid in the body fluid;
c. identifying a subject at risk for developing cardiovascular disease based on the measured level of sphingolipids.

Also disclosed here in is a method of treating cardiovascular disease in a subject, comprising administering a composition elevating sphingolipid levels in a subject.

Also disclosed herein is a method of treating cardiovascular disease in a subject comprising administering a cardiovascular disease drug, such as a beta blocker, an ace inhibitor, and/or a cholesterol lowering medication, such as a statin.

In some forms of the methods one or more steps can be performed by a machine.

In some forms of the methods step b. can be performed by a machine.

In some forms of the methods the machine can be LCMS, or other spectrometric machine, and in certain embodiments the machine can have a computer or be operatively connected to a computer which can do processing, comparing, and analyzing of input biological and physical data.

In some forms of the methods the sphingolipid can be associated with HDL-C or albumin.

In some forms of the methods the body fluid can be blood or plasma.

In some forms of the methods multiple sphingolipids can be measured in the body fluid.

In some forms of the methods the sphingolipids can be measured simultaneously.

In some forms of the methods the sphingolipids can be S1P, DH-S1P or C24:1-ceramide.

In some forms of the methods diagnosing cardiovascular disease comprises comparing the measured level of sphingolipids to a standard.

In some forms of the methods predicting cardiovascular disease comprises comparing the measured level of sphingolipids to a standard.

In some forms of the methods identifying a subject at risk of developing cardiovascular disease comprises comparing the measured level of sphingolipids to a standard.

In some forms of the methods a subject can be diagnosed with cardiovascular disease when the measured level of at least one sphingolipids is at least 10%, 15%, 20%, 25%, 30%, 35%, 40%, 45% or 50% lower than a relevant average standard level of sphingolipids in subjects without cardiovascular disease.

In some forms of the methods a subject can be predicted to have cardiovascular disease when the measured level of at least one sphingolipids is at least 10%, 15%, 20%, 25%, 30%, 35%, 40%, 45% or 50% lower than a relevant average standard level of sphingolipids in subjects without cardiovascular disease.

In some forms of the methods a subject can be identified to be at risk of developing cardiovascular disease when the measured level of at least one sphingolipids is at least 10%, 15%, 20%, 25%, 30%, 35%, 40%, 45% or 50% lower than a relevant average standard level of sphingolipids in subjects without cardiovascular disease.

In some forms of the methods a subject can be diagnosed with cardiovascular disease when the measured level of at least one sphingolipid is at least 25%, 30%, 35%, 40%, 45% or 50% lower than a relevant average standard level of sphingolipids in subjects without cardiovascular disease.

In some forms of the methods a subject can be predicted to have cardiovascular disease when the measured level of at least one sphingolipid is at least 25%, 30%, 35%, 40%, 45% or 50% lower than a relevant average standard level of sphingolipids in subjects without cardiovascular disease.

In some forms of the methods a subject can be identified to be at risk of developing cardiovascular disease when the measured level of at least one sphingolipid is at least 25%, 30%, 35%, 40%, 45% or 50% lower than a relevant average standard level of sphingolipids in subjects without cardiovascular disease.

In some forms of the methods a subject can be diagnosed with cardiovascular disease when the measured level of S1P is at least 0.3 μM, 0.4 μM, 0.5 μM, 0.6 μM, 0.7 μM, 0.8 μM, 0.9 μM or 1.0 μM lower than a relevant average standard level of S1P in subjects without cardiovascular disease.

In some forms of the methods a subject can be diagnosed with cardiovascular disease when the measured level of S1P is at least 0.6 μM, 0.7 μM, 0.8 μM, 0.9 μM or 1.0 μM lower than a relevant average standard level of S1P in subjects without cardiovascular disease.

In some forms of the methods a subject can be diagnosed with cardiovascular disease when the measured level of DH-S1P is at least 0.04 μM, 0.06 μM, 0.08 μM, 0.1 μM, 1.2 μM, 1.4 μM, 1.6 μM, 1.8 μM or 2.0 μM lower than a relevant average standard level of DH-S1P in subjects without cardiovascular disease.

In some forms of the methods a subject can be diagnosed with cardiovascular disease when the measured level of DH-S1P is at least 0.1 μM, 1.2 μM, 1.4 μM, 1.6 μM, 1.8 μM or 2.0 μM lower than a relevant average standard level of DH-S1P in subjects without cardiovascular disease.

In some forms of the methods a subject can be diagnosed with cardiovascular disease when the measured level of C24:1-ceramide is at least 0.04 μM, 0.05 μM, 0.06 μM, 0.07 μM, 0.08 μM, 0.09 μM, or 0.1 μM lower than a relevant average standard level of C24:1-ceramide in subjects without cardiovascular disease.

In some forms of the methods a subject can be diagnosed with cardiovascular disease when the measured level of C24:1-ceramide is at least 0.07 μM, 0.08 μM, 0.09 μM, or 0.1 μM lower than a relevant average standard level of C24:1-ceramide in subjects without cardiovascular disease.

In some forms of the methods a subject can be diagnosed with cardiovascular disease when the [S1P] μM/[apoAI] μM ratio is at least 0.005, 0.007, 0.009, 0.011 or 0.013 lower than a relevant average standard level of [S1P] μM/[apoAI] μM ratio in subjects without cardiovascular disease.

In some forms of the methods a subject can be diagnosed with cardiovascular disease when the [DH-S1P] μM/[apoAI] μM ratio is at least 0.0005, 0.0007, 0.0009, 0.0011 or 0.0013 lower than a relevant average standard level of [DH-S1P] μM/[apoAI] μM ratio in subjects without cardiovascular disease.

In some forms of the methods a subject can be diagnosed with cardiovascular disease the [C24:1-ceramide] μM/[apoAI] μM ratio is at least 0.0005, 0.0007, 0.0009, 0.0011 or 0.0013 lower than a relevant average standard level of [C24:1-ceramide] μM/[apoAI] μM ratio in subjects without cardiovascular disease.

In some forms of the methods a subject can be predicted to have cardiovascular disease when the measured level of S1P is at least 0.3 μM, 0.4 μM, 0.5 μM, 0.6 μM, 0.7 μM, 0.8 μM, 0.9 μM or 1.0 μM lower than a relevant average standard level of S1P in subjects without cardiovascular disease.

In some forms of the methods a subject can be predicted to have cardiovascular disease when the measured level of S1P is at least 0.6 μM, 0.7 μM, 0.8 μM, 0.9 μM or 1.0 μM lower than a relevant average standard level of S1P in subjects without cardiovascular disease.

In some forms of the methods a subject can be predicted to have cardiovascular disease when the measured level of DH-S1P is at least 0.04 μM, 0.06 μM, 0.08 μM, 0.1 μM, 1.2 μM, 1.4 μM, 1.6 μM, 1.8 μM or 2.0 μM lower than a relevant average standard level of DH-S1P in subjects without cardiovascular disease.

In some forms of the methods a subject can be predicted to have cardiovascular disease when the measured level of DH-S1P is at least 0.1 μM, 1.2 μM, 1.4 μM, 1.6 μM, 1.8 μM or 2.0 μM lower than a relevant average standard level of DH-S1P in subjects without cardiovascular disease.

In some forms of the methods a subject can be predicted to have cardiovascular disease when the measured level of C24:1-ceramide is at least 0.04 μM, 0.05 μM, 0.06 μM, 0.07 μM, 0.08 μM, 0.09 μM, or 0.1 μM lower than a relevant average standard level of C24:1-ceramide in subjects without cardiovascular disease.

In some forms of the methods a subject can be predicted to have cardiovascular disease when the measured level of C24:1-ceramide is at least 0.07 μM, 0.08 μM, 0.09 μM, or 0.1 μM lower than a relevant average standard level of C24:1-ceramide in subjects without cardiovascular disease.

In some forms of the methods a subject can be predicted to have cardiovascular disease when the [S1P] μM/[apoAI] μM ratio is at least 0.005, 0.007, 0.009, 0.011 or 0.013 lower than a relevant average standard level of [S1P] μM/[apoAI] μM ratio in subjects without cardiovascular disease.

In some forms of the methods a subject can be predicted to have cardiovascular disease when the [DH-S1P] μM/[apoAI] μM ratio is at least 0.0005, 0.0007, 0.0009, 0.0011 or 0.0013 lower than a relevant average standard level of [DH-S1P] μM/[apoAI] μM ratio in subjects without cardiovascular disease.

In some forms of the methods a subject can be predicted to have cardiovascular disease the [C24:1-ceramide] μM/[apoAI] μM ratio is at least 0.0005, 0.0007, 0.0009, 0.0011 or 0.0013 lower than a relevant average standard level of [C24:1-ceramide] μM/[apoAI] μM ratio in subjects without cardiovascular disease.

In some forms of the methods a subject can be identified to be at risk of developing cardiovascular disease when the measured level of S1P is at least 0.3 μM, 0.4 μM, 0.5 μM, 0.6 μM, 0.7 μM, 0.8 μM, 0.9 μM or 1.0 μM lower than a relevant average standard level of S1P in subjects without cardiovascular disease.

In some forms of the methods a subject can be identified to be at risk of developing cardiovascular disease when the measured level of S1P is at least 0.6 μM, 0.7 μM, 0.8 μM, 0.9 μM or 1.0 μM lower than a relevant average standard level of S1P in subjects without cardiovascular disease.

In some forms of the methods a subject can be identified to be at risk of developing cardiovascular disease when the measured level of DH-S1P is at least 0.04 μM, 0.06 μM, 0.08 μM, 0.1 μM, 1.2 μM, 1.4 μM, 1.6 μM, 1.8 μM or 2.0 μM lower than a relevant average standard level of DH-S1P in subjects without cardiovascular disease.

In some forms of the methods a subject can be identified to be at risk of developing cardiovascular disease when the measured level of DH-S1P is at least 0.1 μM, 1.2 μM, 1.4 μM, 1.6 μM, 1.8 μM or 2.0 μM lower than a relevant average standard level of DH-S1P in subjects without cardiovascular disease.

In some forms of the methods a subject can be identified to be at risk of developing cardiovascular disease when the measured level of C24:1-ceramide is at least 0.04 μM, 0.05 μM, 0.06 μM, 0.07 μM, 0.08 μM, 0.09 μM, or 0.1 μM lower than a relevant average standard level of C24:1-ceramide in subjects without cardiovascular disease.

In some forms of the methods a subject can be identified to be at risk of developing cardiovascular disease when the measured level of C24:1-ceramide is at least 0.07 μM, 0.08 μM, 0.09 μM, or 0.1 μM lower than a relevant average standard level of C24:1-ceramide in subjects without cardiovascular disease.

In some forms of the methods a subject can be identified to be at risk of developing cardiovascular disease when the [S1P] μM/[apoAI] μM ratio is at least 0.005, 0.007, 0.009, 0.011 or 0.013 lower than a relevant average standard level of [S1P] μM/[apoAI] μM ratio in subjects without cardiovascular disease.

In some forms of the methods a subject can be identified to be at risk of developing cardiovascular disease when the [DH-S1P] μM/[apoAI] μM ratio is at least 0.0005, 0.0007, 0.0009, 0.0011 or 0.0013 lower than a relevant average standard level of [DH-S1P] μM/[apoAI] μM ratio in subjects without cardiovascular disease.

In some forms of the methods a subject can be identified to be at risk of developing cardiovascular disease when the [C24:1-ceramide] μM/[apoAI] μM ratio is at least 0.0005, 0.0007, 0.0009, 0.0011 or 0.0013 lower than a relevant average standard level of [C24:1-ceramide] μM/[apoAI] μM ratio in subjects without cardiovascular disease.

In some forms of the methods the cardiovascular disease can be ischemic heart disease.

In some forms of the methods the ischemic heart disease can be atherosclerosis.

In some forms of the methods the subject could have no traditional risk factors of having or developing cardiovascular disease.

In some forms of the methods the subject can have high HDL-C.

In some forms of the methods can further comprise reducing LDL-C level from the body fluid.

In some forms of the methods the subject does not have conventional risk factors associated with cardiovascular disease.

In some forms of the methods the conventional risk factor can be elevated LDL-C or low HDL-C.

In some forms of the methods the subject is in need of treatment for cardiovascular disease.

In some forms of the methods the subject is monitored for cardiovascular disease.

In some forms of the methods the subject diagnosed with cardiovascular disease.

In some forms of the methods the sphingolipid level can be S1P, DH-S1P or C24 ceramide.

In some forms of the methods the composition can comprise S1P, DH-S1P or C24 ceramide.

In some forms of the methods the composition can comprise a statin.

### V. BRIEF DESCRIPTION OF DRAWINGS

FIG. 1 shows that an inverse correlation exists between the occurrence of IHD and levels of S1P and DH-S1P in HDL-containing fractions from CCHS subject serum. S1P (A) DH-S1P (B) and C24.1 ceramide (C) levels were measured by blinded LC-MS-MS analysis of 55 HDL-containing preparations from CCHS individuals with high HDL and no evidence of IHD, 53 samples from individuals with high HDL and evidence of IHD, 54 samples from individuals with low HDL and no evidence of IHD and 42 samples from individuals with low HDL and evidence of IHD. For the box-and-whisker diagrams, the boxes correspond to the interquartile range (IQR). The horizontal bar within the box is drawn at the height of the median. The whiskers indicate the range of the data within 1.5×IQR with outliers indicated as circles.

FIG. 2 shows that an inverse correlation exists between the occurrence of IHD and levels of S1P and DH-S1P in HDL-containing serum fractions assessed relative to apoA-I content. Levels of S1P (A) and DH-S1P (B) and C24:1-ceramide (C) were measured by blinded LC-MS-MS in HDL-containing preparations from 204 CCHS serum samples as defined in Table 2. Levels of apo-AI in samples were quantified by immunoturbidometric assay using a Cobas Fara analyzer. The horizontal bar within the box is drawn at the height of the median. The whiskers indicate the range of the data within 1.5×IQR with outliers indicated as circles.

FIG. 3 shows that HDL-enhanced transendothelial electrical resistance is inhibited by pertussis toxin and S1P1 antagonists. Confluent endothelial monolayers were grown under serum-free conditions until a minimal TEER plateau had been reached. The cells were then incubated with S1P or HDL in the presence or absence of PTX (A and B) or S1P1 antagonists (C and D). In A and B, S1P was used at 833 nM, HDL at 1000 μg/ml (containing 400 nM S1P) and PTX at 1 μg/ml. In C and D, S1P was used at 250 nM, HDL at 621 μg/ml (containing 250 nM S1P), the S1P1 antagonist 857390 at 10 μM and the S1P1/S1P3 antagonist VPC23019 at 10 μM. As controls, monolayers were treated with BSA-containing serum free medium (SFM) plus or minus vehicle buffer. Each of the TEER tracings shown is an average of two replicate wells and representative of three independent experiments. Impedance values were normalized by dividing each value by the level of impedance measured just prior to the addition of effectors.

FIG. 4 shows that HDL stimulates Erk1/2 and Akt activation in endothelial cells. The effect of S1P and HDL on activation of Erk1/2 (A-D) and Akt (E-H) in HUVECs was determined by multiplex bead array assay. In A-D, the values for the fold difference in Erk1/2 phosphorylation were derived from the level of phosphoErk1/2 fluorescence in S1P or HDL treated cells divided by the level of phosphoErk1/2 fluorescence in control cells. In E-G, the values for the fold increase in Akt phosphorylation were derived from the level of phosphoAkt fluorescence in cells treated with S1P or HDL divided by the level of phosphoAkt fluorescence measured in control cells. The data depicted in panels A, C, E and G is based on treating HUVECs for the indicated times with 833 nM S1P or 333 μg/ml HDL (containing 133 nM S1P). The data depicted in panels B, D, F and H is based on treating HUVECs with indicated concentrations of S1P or HDL for 3 min. The level of S1P in the 3-fold dilutions of HDL tested in panel H ranged from 12-337 nM. Data are shown from a representative experiment.

FIG. 5 shows HDL activation of Erk1/2 and Akt in HUVECs is inhibited by pertussis toxin and S1P1 antagonists. In A and B, pertussis toxin (PTX, 100 ng/ml) or the PTX buffer was added to endothelial cell basal medium (EBM) during the final 12 h of serum starvation. BSA, S1P (200 nM) or HDL (containing 200 nM S1P) were then added to the medium and allowed to incubate with the cells for 3 min. In C and D, the S1P1 antagonist 857390 or the S1P1/S1P3 antagonist or vehicle were added to the medium 15 min prior to addition of S1P or HDL and allowed to incubate with the cells for 3 min. The graphed values were derived from the level of phosphoErk1/2 or phosphoAkt fluorescence in S1P- or HDL-treated cells divided by the level of phosphoErk1/2 or phosphoAkt fluorescence in BSA treated cells. Data are shown from representative experiments, antagonists and inhibitor experiments were performed 2-4 times (e.g. PTX, n=3; S1P1 antagonist, n=4; S1P1/S1P3 antagonist, n=2). Each data point is an average from two independent wells.

FIG. 6 shows that an inverse correlation exists between IHD and levels of S1P and dihydro-S1P in serum samples from Copenhagen City Heart Study subjects. Levels of S1P (A) and dihydro-S1P (B) measured by blinded LC-MS-MS in samples from individuals with high HDL having ischemic heart disease (IHD) as compared to individuals with high HDL having no evidence of IHD. The graphs are based on data from a blinded LC-MS-MS analysis of 204 CCHS serum samples that had been freed of apoB-containing particles by dextran-sulfate precipitation and thus can be considered LDL poor, HDL-containing preparations. The analysis was performed on 55 samples from individuals with high HDL (♀:≧73.5 mg/dL; ♂:≧61.9 mg/dL) and no evidence of IHD, 53 samples from individuals with high HDL and evidence of IHD, 54 samples from individuals with low HDL (♀:≦38.7 mg/dL; ♂:≦34.1 mg/dL) and no evidence of IHD and 42 samples from individuals with low HDL and evidence of IHD (see Table 3).

FIG. 7 shows that serum levels of S1P and dihydro-S1P as a function of apoA-I levels inversely correlate with IHD. Levels of S1P (A) and dihydro-S1P (B) were measured by blinded LC-MS-MS in samples from 204 CCHS serum samples as described in Table I. Levels of apo-AI in serum samples from Copenhagen City Heart Study subjects were quantified using an immunoturbidometric assay using a Cobas Fara analyzer (Roche Diagnostic Systems, Inc.).

FIG. 8 shows that HDL enhances transendothelial electrical resistance. A minimal TEER plateau was reached within ˜24 h of replacing the culture medium of confluent endothelial cells with serum free medium. The monolayers were then incubated with varying concentrations S1P (A) or HDL (B). In B, the concentration of S1P in the HDL ranged from 5-403 nM. The TEER tracings represent mean data from 3 independent experiments each with 2 replicates per condition. As a control, monolayers were treated with 40 μg/ml BSA, a concentration corresponding to the amount used for the highest concentration of S1P tested. Impedance values were normalized by dividing each value by the level of impedance measured just prior to the addition of effectors.

FIG. 9 shows that synthetic HDL containing S1P enhances transendothelial electrical resistance. A minimal TEER plateau was reached within ˜24 h of replacing the culture medium of confluent endothelial cells with serum free medium. The monolayers were then incubated with native HDL (250 nM S1P), synthetic HDL containing S1P (250 nM S1P), synthetic HDL lacking S1P or delipidated bovine serum albumin LC-MS-MS analysis showed that the synthetic HDL minus S1P preparation contained small amounts of S1P, most likely derived from the apoA-I preparation. The level of S1P in the synthetic HDL minus S1P tested in this assay was 7 nM.

FIG. 10 shows the percentage of Subjects with IHD versus S1P Quartile.

FIG. 11 shows the percentage of Subjects with IHD versus DH-S1P Quartile.

FIG. 12 shows the HDL and reconstituted-HDL enhance transendothelial electrical resistance in a manner related to associated S1P levels. S1P-augmented HDL was prepared by preincubation of native HDL with S1P followed by dialysis against 0.03 mM EDTA in Dulbecco's PBS to remove free S1P. For reconstituted-HDL (rHDL) preparations, lipids were extracted from native HDL using diethyl ether and reconstituted using a 1:100 molar ratio of delipidated HDL:POPC according to the cholate dialysis method of Matz and Jonas. (JBC 257(8) 1982). A minimal TEER plateau was reached within ˜24 h of replacing culture medium of confluent ECs with serum free medium. Monolayers were incubated with 250 μg/ml S1P-augmented HDL (A) or rHDL (B) containing varying amounts of S1P as indicated in figure legend. Impedance values were normalized by dividing each value by the level of impedance measured just prior to the addition of effectors.

### VI. DETAILED DESCRIPTION OF THE INVENTION

Described herein are aspects of lipoprotein biology, i.e., that lipoproteins carry bioactive sphingolipids and that the sphingolipid composition of lipoproteins is a major factor in the process by which lipoproteins impact the etiology of cardiovascular disease. At least one lipoprotein-associated sphingolipid, S1P, is well known to elicit an array of vascular responses, many of which can be considered as cardioprotective. Furthermore, many of the cardioprotective effects of HDL as described herein can be attributed to its S1P cargo. Now through analysis of large numbers of blood samples from human subjects, it is established that levels of S1P as well as two other sphingolipids have highly significant inverse correlations with the occurrence of IHD. This is evidence that in addition to cholesterol, sphingolipids is risk factors for IHD as well as targets for therapeutic intervention. As described herein it is establish that low levels of lipoprotein-associated sphingolipids are IHD risk factors, then therapies that increase specific plasma HDL-sphingolipid levels can decrease the risk for IHD.

Evidence indicates that high blood levels of HDL are cardioprotective, however, there are individuals with very high levels of HDL and no other known risk factors of cardiovascular disease (such as increased levels of LDL) that have heart disease indicating that qualitative differences might exist in HDL particles which make them functionally different with respect to cardioprotection. It is shown herein that individuals with high levels of HDL and clinical evidence of heart disease have low levels of sphingosine-1-phosphate (S1P), a lipid normally carried on HDL that has been shown to have a number of beneficial effects on the cardiovascular system. It is shown herein that HDL-associated S1P levels inversely correlate with cardiovascular disease or risk of developing cardiovascular disease and that HDL particles with lower than normal levels of S1P are dysfunctional with respect to cardioprotective-related activities.

Levels of sphingolipids in HDL inversely correlate with risk of cardiovascular disease. Liquid chromatography-mass spectroscopy (LCMS) was used to measure levels of S1P and other related sphingolipids in plasma lipoprotein samples from a group of individuals with high HDL (>80 mg/dl) with and without ischemic heart disease (IHD) as well as from individuals with low HDL with and without IHD. The samples for these studies were obtained from the Copenhagen City Heart Study. The Copenhagen City Heart Study population comprises samples from ˜20,000 clinically characterized individuals 20 years of age and older. From this collection of samples, a set of samples was obtained from groups of patients with high HDL and IHD and low HDL with IHD. The samples were selected from an equal number of age-matched controls for each (i.e., high HDL with no evidence of cardiovascular disease and low HDL no evidence of cardiovascular disease) (see Table 1). A number of compositional analyses were performed on the samples including measurements of levels of TG, PL, apoA-I, apoA-II and apoE HDL subfractions. For LCMS analysis, the serum samples were freed of apoB-containing particles by precipitation and thus can be considered LDL poor, HDL-containing preparations.

TABLE 1

| | With IHD | | No IHD | |
|---|---|---|---|---|
| | High HDL | Low HDL | High HDL | Low HDL |
| | (n = 53) | (n = 42) | (n = 55) | (n = 54) |
| | Group 1 | Group 2 | Group 3 | Group 4 |
| Age, years | 63.1 ± 10.3 | 61.5 ± 9.3 | 62.6 ± 10.3 | 62.7 ± 9.6 |
| Total cholesterol, mg/dL | 208.1 ± 25.7 | 182.3 ± 29.4 | 207.3 ± 31.2 | 166.8 ± 31.1 |
| High density lipoprotein-C, mg/dL | 78.4 ± 14.3 | 32.4 ± 5.2 | 80.5 ± 14.1 | 33.6 ± 5.7 |
| Low density lipoprotein-C, mg/dL | 113.4 ± 26.3 | 120.9 ± 30.6 | 118.8 ± 28.0 | 117.7 ± 25.6 |
| Triglycerides, mg/dL | 82.1 ± 30.0 | 104.9 ± 31.1 | 74.1 ± 25.2 | 107.8 ± 29.4 |
| Body mass index, kg/m² | 24.8 ± 4.2 | 26.0 ± 3.5 | 23.6 ± 3.1 | 28.0 ± 5.0 |
| Smokers, % | 27.1 | 26.8 | 42.6 | 33.3 |
| Diabetes mellitus, % | 7.7 | 14.3 | 5.5 | 7.4 |

Table 1 shows the characteristics of participants with and without IHD from the Copenhagen University Hospital and The Copenhagen City Heart Study. All values are original measurements from the above-mentioned studies. Selection and matching for the present study were based on these values. All individuals had LDL-C <160 mg/dL, triglycerides <150 mg/dL, and none were treated with LDL-C-lowering medications. Group 1: Females n = 16 Males n = 37; Group 2: Females n = 13 Males n = 29; Group 3: Females n = 16 Males n = 39; Group 4: Females n = 15 Males n = 39. Group 1 had high HDL-C (>90th percentile) and verified IHD; this group was compared with Group 3 without IHD, but matched by age, sex, and similar HDL-C levels (>90th percentile HDL-C levels for Group 1 and 3 individuals were females: ≧73.5 mg/dl; males: ≧61.9 mg/dL). Group 2 had low HDL-C (<10th percentile) and verified IHD; this group was compared with Group 4 without IHD, but matched by age, sex, and similar HDL-C levels (<10th percentile HDL-C levels for Group 2 and 4 individuals were females: ≦38.7 mg/dL; males: ≦34.1 mg/dL). All individuals without IHD were selected from The Copenhagen City Heart Study's 4th examination. Patients with IHD were selected from individuals referred to the Copenhagen University Hospital, Rigshospitalet, Denmark for coronary angiography.

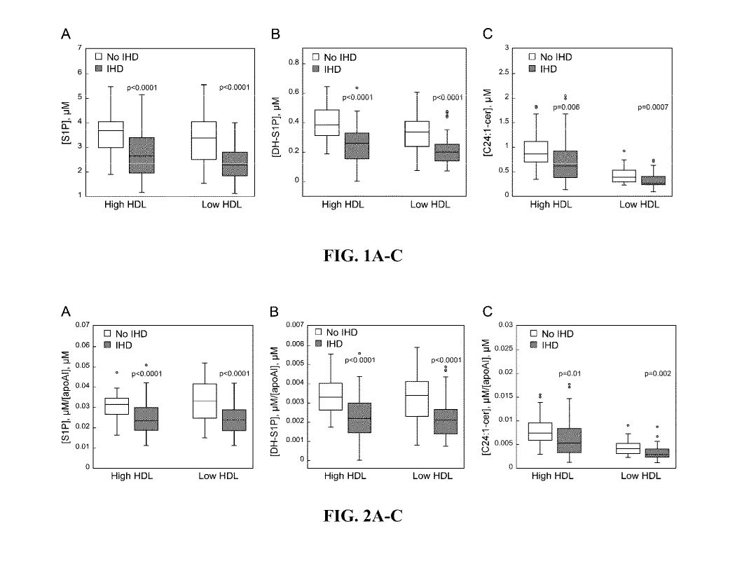

Statistical analysis of the LC-MS-MS data (see Table 2) showed that S1P and DH-S1P levels were significantly lower (p<0.0001) in the HDL-containing serum fractions from individuals with high HDL-C having IHD as compared to individuals with high HDL-C having no evidence of IHD (FIGS. 1A and B). Furthermore, S1P and DH-S1P levels were significantly lower (p<0.0001) in HDL-containing fractions from individuals with low HDL-C and having IHD as compared to individuals with low HDL-C having no evidence of IHD (FIGS. 1A and B).

TABLE 2

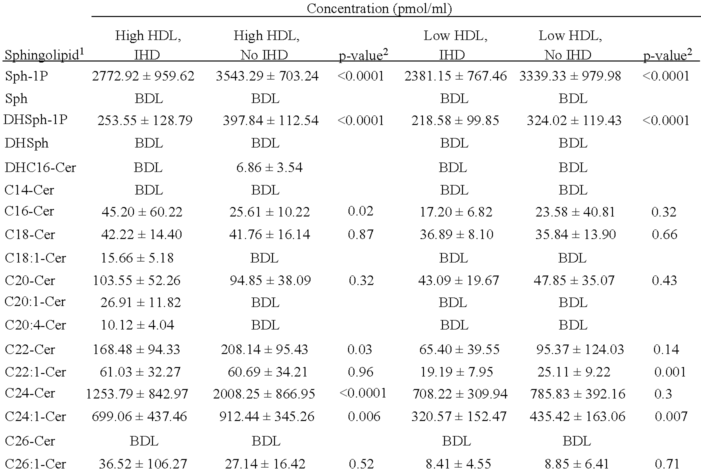

¹Abbreviations used: Sph, sphingosine; DH, dihydro; Cer, ceramide; P, phosphate; BDL, below detection limit.
², p-values were calculated using a one-way ANOVA test conducted at level of significance 0.05.

Among the other sphingolipids analyzed in the HDL-containing fractions from the CCHS subjects, C24:1-ceramide levels were significantly lower (p=0.006) in HDL-containing fractions from individuals with high HDL-C having IHD as compared to individuals with high HDL-C having no evidence of IHD (FIG. 1C). Furthermore, C24:1-ceramide levels were significantly lower (p=0.0007) in samples from individuals with low HDL-C and having IHD as compared to individuals with low HDL-C having no evidence of IHD (FIG. 1C).

When S1P, DH-S1P and C24:1-ceramide levels were assessed relative to the concentration of apoA-I in the samples, the ratio of the concentration of these sphingolipids to apoA-1 concentration were all significantly lower (p<0.05) in samples from individuals with high HDL-C having IHD as compared to individuals with high HDL-C having no evidence of IHD (FIG. 2A-C). Furthermore, these ratios were also significantly lower (p<0.05) in samples from individuals with low HDL-C and having IHD as compared to individuals with low HDL-C having no evidence of IHD (FIG. 2A-C).

### 1. Cardiovascular Disease

Cardiovascular diseases (CVDs) are a leading cause of disability and death in the developed world, resulting in more premature deaths than any other illness. Unsurprisingly, treatment of CVD represents a very high cost burden to any healthcare system. Accordingly, there is tremendous social and political pressure to develop earlier and more reliable diagnostic tests to assist in the detection, treatment, and prevention of CVD.

Cardiovascular disease can be any disorder that affects the heart or the vasculature. CVD, as the term is used herein, can refer to any disease that affects the cardiovascular system, in particular, CVD conditions include, but are not limited to: coronary (ischemic) heart disease; angina pectoris; arrhythmia; cardiac fibrosis; congenital cardiovascular disease; coronary artery disease; peripheral vascular disease; dilated cardiomyopathy; heart attack (myocardial infarction); cerebrovascular disease (stroke); atherosclerosis; heart failure; hypertrophic cardiomyopathy; systemic hypertension from any cause; edematous disorders caused by liver or renal disease; mitral regurgitation; myocardial tumors; myocarditis; rheumatic fever; Kawasaki disease; Takaysu arteritis; cor pulmonale; primary pulmonary hypertension; amyloidosis; hemachromatosis; toxic effects on the heart due to poisoning; Chaga's disease; heart transplantation; cardiac rejection after heart transplantation; cardiomyopathy of chachexia; arrhythmogenic right ventricular dysplasia; cardiomyopathy of pregnancy; Marfan Syndrome; Turner Syndrome; Loeys-Dietz Syndrome; familial biscuspid aortic valve or any inherited disorder of the heart or vasculature, or combinations thereof. These and other CVD conditions have related causes, mechanisms, and treatments. In practice, CVD can be treated by cardiologists, thoracic surgeons, vascular surgeons, neurologists, and interventional radiologists, depending on the organ system that is being treated. There is considerable overlap in the specialties, and it is common for certain procedures to be performed by different types of specialists in the same hospital.

Currently, several risk factors are used by medical professionals to assess an individual's risk of developing or having CVD and to identify individuals at high risk. Major risk factors for cardiovascular disease include age, hypertension, family history of premature CVD (genetic predisposition), smoking, high total cholesterol, elevated LDL, low HDL cholesterol, obesity and diabetes, C-reactive protein, blood levels of myeloperoxidase (See commonly assigned U.S. patent application Ser. No. 10/039,753, which is specifically incorporated herein by reference in its entirety) or modified apolipoprotein A-I (See commonly assigned, U.S. application Ser. No. 11/005,563, which is specifically incorporated herein by reference in its entirety.) The major risk factors for CVD are typically used together by physicians in a risk prediction algorithm to target those individuals who are most likely to benefit from treatment for CVD. In addition, CVD can develop and CVD complications can occur in individuals with apparently low to moderate risk profiles, as determined using currently known risk factors.

A low-fat diet and exercise are recommended to prevent CVD. In addition, a number of therapeutic agents may be prescribed by medical professionals to those individuals who are known to be at risk for developing or having CVD. These include lipid-lowering agents that reduce blood levels of cholesterol and trigylcerides, agents that normalize blood pressure, agents, such as aspirin or platelet ADP receptor antatoginist (e.g., clopidogrel and ticlopidine), that prevent activation of platelets and decrease vascular inflammation, and pleotrophic agents such as peroxisome proliferator activated receptor (PPAR) agonists, with broad-ranging metabolic effects that reduce inflammation, promote insulin sensitization, improve vascular function, and correct lipid abnormalities. More aggressive therapy, such as administration of multiple medications or surgical intervention may be used in those individuals who are at high risk. Since CVD therapies may have adverse side effects, it is desirable to have additional agents for treating individuals who have or are at risk of having or developing CVD.

### 2. HDL

There are several main classes of plasma transporters, which carry and enhance the exchange of lipids in the circulation and between plasma and cells. These include the chylomicrons (CM), the very low—density lipoproteins (VLDL), the intermediate density lipoproteins (IDL), the low—density lipoproteins (LDL) and HDL. A number of others exist (lipoprotein a, subtypes of the main classes), though not routinely measured.

Low HDL-cholesterol (HDL-C), high LDL-C and high plasma triglycerides (Tg) embody a dyslipidemia, common for atherosclerosis, T2D, obesity and MBO.

HDL represents one of the main lipoprotein carriers of cholesterol. Low HDL-C levels characterize about 10% of the general population (Sampietro T et al, 2005). Furthermore, low HDL concentration represents the most frequent dyslipidemia in patients with coronary artery disease (CAD) (Sampietro T, et al, 2005).

Despite of the existence of a number of drugs successfully reducing LDL plasma availability, the following reduction of cardiovascular risk does not prove to be enough sufficient. A number of clinical studies have been aiming to determine whether aggressive lowering of LDL-C beyond the currently accepted guidelines would result in further reduction of cardiovascular events (Cannon C P, et al, 2004; Waters D D, et al, 2004). The results from some of those studies are still pending, while others such as the PROVE IT-TIMI 22 (Cannon, C. P., et al., 2004) have shown certain benefits of aggressive lowering of LDL, which, however, leave remarkably high residual cardiovascular disease (CVD) occurrence.

HDL is traditionally an independent predictor of the risk of cardiovascular disease (Castelli W P et al, 1986, Salonen J T et al, 1991). Already in 1977 it was shown that CAD patients have 35% lower HDL-C levels than controls and those with lowered HDL have been exposed to three times higher likelihood of developing CAD than those with elevated LDL-C (Miller N E et al, 1977). Low HDL-C was observed to be the most common lipid abnormality in men with coronary artery disease (Genest J J et al, 1991). According to the first large-scale prospective trial to study the effect of raising HDL-C on CAD incidence (the Helsinki Heart Study), 11% increase in HDL-C levels was independently associated with a 34% reduction in CAD events (Manninen V et al, 1992). A number of other clinical studies have confirmed a significantly reduced incidence of coronary events after an increase in HDL-C concentration (Alberti K G 1998; Frick M H et al, 1987; Rubins H B et al, 1999). Thus elevating the low HDL-C levels independently or in combination with a decreasing of the high LDL-C state represents a frontier in the treatment and prevention of CVD. However, people with high levels of HDL-C can also develop CVD, thus high HDL-C levels is not always a predictable indicator of low risk of developing CVD. As described herein sphingolipids associated with HDL-C have been shown to impact the cardioprotective properties of HDL-C and thus should also be measured when analyzing people for CVD. Low levels of sphingolipids associated with HDL-C have been shown to decrease the cardioprotective properties of HDL-C.

### 3. Sphingolipids

Sphingolipids generally are composed of a long-chain (sphingoid) base (sphingosine, sphinganine, 4-hydroxysphinganine, or a related compound) as the backbone moiety (Karlsson, K. A. Chem. Phys. Lipids, 5: 6-43, 1970), which is usually modified by an amide-linked long-chain fatty acid (for ceramides), and a head group at position 1. Over 300 classes of sphingolipids are known, most of which have head groups with simple to complex carbohydrates (see Merrill & Sweeley, New Comprehensive Biochemistry: Biochemistry of Lipids, Lipoproteins, and Membranes, (Vance, D. E. & Vance, J. E., eds.), pp. 309-338, Elsevier Science, Amsterdam, 1996).

It is a common misconception from the names of these compounds (e.g., ceramide, sphingomyelin, gangliosides, etc.) that sphingolipids are only found in neuronal tissues. In fact, sphingolipids are major constituents of all eukaryotic (and some prokaryotic) organisms, including plants (Lynch, D. V., Lipid Metabolism in Plants (T. S. Moore, Jr., ed.), pp. 285-308, CRC Press, Boca Raton, Fla. 1993). This nomenclature merely reflects their initial discovery in brain tissues by classic studies a century ago (Thudichum, J. L. W., A Treatise on the Chemical Constitution of Brain, Bailliere, Tindall & Cox, (London) 1884).

Mammalian sphingolipid compounds typically vary in the presence or absence of the 4,5-trans-double bond (for example, sphingosine has a double bond whereas sphinganine (also referred to as dihydrosphingosine) does not); (ii) double bond (s) at other positions, such as position 8; (iii) a hydroxyl group at position 4 (D-1-hydroxysphinganine, also called “phytosphingosine”) or elsewhere (Robson et al., J. Lipid Res. 35: 2060-2068, 1994); (iv) methyl group (s) on the alkyl side chain or on the amino group, such as N,N,-dimethylsphingosine; and (v) acylation of the amino group (for example ceramide (also referred to as N-acylsphingosine), and dihydroceramide (also referred to as N-acyl-sphinganine)). The 4-hydroxysphinganines are the major long-chain bases of yeast (Wells, G. B. and Lester, R. L., J. Biol. Chem., 258: 10200-10203, 1983), plants (Lynch, D. V., Lipid Metabolism in Plants (T. S. Moore, Jr., ed.), pp. 285-308, CRC Press, Boca Raton, Fla. 1993), and fungi (Merrill et al., Fungal Lipids (R. Prasad and M. Ghanoum, eds.), CRC Press, Boca Raton, Fla., 1995a), but are also made by mammals (Crossman and Hirschberg, J. Biol. Chem., 252: 5815-5819, 1977). Other modifications of the long-chain base backbone include phosphorylation at the hydroxyl oxygen of carbon 1 (Buehrer and Bell, Adv. Lipid Res. 6: 59-67, 1993), and acylation (Merrill and Wang, Methods Enzymol., 209: 427-437, 1992) (Igarashi and Hakomori, Biochim. Biophys. Res. Commun. 164: 1411-1416, 1989; Felding-Habermann et al., Biochemistry 29: 6314-6322, 1990) of the amino group.

Each of these compounds can be found in various alkyl chain lengths, with 18 carbons predominating in most sphingolipids, but other homologs can constitute a major portion of specific sphingolipid (as exemplified by the large amounts of C₂₀ sphingosine in brain gangliosides) (Valsecchi et al., J. Neurochem., 60: 193-196, 1993) and in different sources (e.g., C₁₆ sphingosine is a substantial component of milk sphingomyelin) (Morrison, Biochim Biophys. Acta., 176: 537-546, 1969).

The lysosphingolipid, sphingosine 1-phosphate (S1P), is a component of human plasma (Yatomi, Y., et al., Journal of Biochemistry 121:969-973). Approximately 65% of the S1P in blood is associated with the lipoproteins LDL, VLDL and HDL, with the bulk of lipoprotein-associated S1P (˜85%) bound to HDL (Murata, N., et al., Biochem J 352 Pt 3: 890-815). Findings from a growing number of studies indicate that S1P is a mediator of many of the cardiovascular effects of HDL including the ability to promote vasodilation, vasoconstriction, angiogenesis, endothelial barrier function, protect against ischemia/reperfusion injury and inhibit/reverse of atherosclerosis (Argraves, K. m., et al., J Biol Chem 283:25074-25081; Argraves, K. M., J Lipid Res 48: 2325-2333). These latter cardioprotective effects of HDL have been shown to involve S1P-mediated suppression of inflammatory processes including reduction of endothelial expression of monocyte and lymphocyte adhesion molecules, decreased recruitment of polymorphonuclear cells to sites of infarction as well as augmented endothelial barrier activity and blocking of cardiomyocyte apoptosis following myocardial infarction (Argraves, K. m., et al., J Biol Chem 283:25074-25081; Argraves, K. M., J Lipid Res 48: 2325-2333). These findings highlight the need for further investigation of the physiological significance of lipoproteins as carriers of S1P, particularly to determine if alterations in plasma levels of HDL-associated S1P underlie cardiovascular pathologies linked with dyslipidemia.

Sphingolipid metabolites, namely ceramide (Cer) and sphingosine 1-phosphate (S1P), are increasingly being recognized for their role as signaling molecules. While there is much known about that bioactivities of S1P, particularly in the context of vascular biology, little is known as to the biological actions of DH-S1P and C24:1-ceramide. Recently, DH-S1P has been shown to mediate activation ERK1/2 and induction of matrix metalloproteinase 1 (MMP1) expression in dermal fibroblasts (Bu et al., 2006, Faseb J 20: 184-186). These effects were not reproduced by S1P and the receptor responsible is yet to be identified. MMP1 is believed to play an important role in the pathogenesis of atherosclerosis. Findings from mouse studies indicates that MMP1 can inhibit atherosclerosis (Lemaitre et al., 2001, J Clin Invest 107: 1227-1234) and recent human studies show that persons homozygous for a transcriptionally overactive allele of the MMP1 have a reduced risk of coronary heart disease (Ye et al., European heart journal 24:1668-1671). Thus, lower levels of HDL-associated DH-S1P might be predicted to reduce MMP1 expression and consequently its atheroprotective effects. Therefore, therapies that act to increase plasma DH-S1P levels into the range associated with decreased risk for IHD should be patent protected.

The lysosphingolipid, sphingosine 1-phosphate (S1P) is carried in the blood in association with lipoproteins, predominantly high density lipoproteins (HDL). Evidence indicates that many of the cardiovascular effects of HDL may be attributable to its S1P cargo. Disclosed herein it is shown that levels of S1P and related sphingolipids in the HDL-containing fraction of human serum inversely correlate with occurrence of ischemic heart disease (IHD). Liquid chromatography-mass spectrometry was used to measure S1P levels in an HDL containing fraction of serum (depleted of LDL and VLDL) from 204 subjects in the Copenhagen City Heart Study (CCHS). The study group consisted of individuals having high serum HDL cholesterol (HDL-C) (females:≧73.5 mg/dL; males:≧61.9 mg/dL) and verified IHD; subjects with high HDL-C and no IHD; individuals with low HDL-C (females:≦38.7 mg/dL; males:≦34.1 mg/dL) and IHD, and subjects with low HDL-C and no IHD. The results show a highly significant inverse relationship between the level of S1P in the HDL-containing fraction of serum and the occurrence of IHD. Furthermore, a similar inverse relationship with IHD was also observed for two other sphingolipids, dihydro-S1P and C24:1-ceramide, in the HDL-containing fraction of serum. These findings indicate that compositional differences in sphingolipid content of HDL might be important in deciphering the putative protective role of HDL in IHD.

An inverse correlation exists between levels of HDL-associate DH-S1P and ischemic heart disease (IHD). As disclosed herein a group IHD subjects had a positive history of angina pectoris plus at least one of the following criteria: stenosis/atherosclerosis on coronary angiography, a previous MI, or significant myocardial ischemia on a bicycle exercise electrocardiography test. Thus, the HDL-associate DH-S1P levels inversely correlate with risk of generalized cardiovascular disease that includes stroke.

Epidemiological data from the Framingham Heart Study (Gordon, T., et al., The American journal of medicine 62: 707-714; Gordon, T., et al., Archives of internal medicine 141: 1128-1131) and other prospective studies (Gordon, D. J., et al., Circulation 79: 8-15) demonstrate that high blood levels of HDL cholesterol (HDL-C) are inversely associated with risk for cardiovascular disease. However, some individuals with high HDL-C together with normal LDL-C develop cardiovascular disease (Ansell, B. J., et al., Circulation 108: 2751-2756). This indicates that the HDLs in these individuals might be dysfunctional as anti-atherogenic particles, perhaps due to quantitative abnormalities with respect to S1P content. Disclosed herein are methods measuring levels of S1P and related sphingolipids in HDL-containing fractions from groups of individuals having high and low HDL-C, with and without evidence of ischemic heart disease.

### 4. Sphingolipids Measurements

Lysosphingolipids can be measured by various techniques from a range of body fluids and extracts of tissues and cells. Liquid chromatography mass spectroscopy (LCMS) as described by Pettus et al (Pettus B J, et al., Rapid Commun Mass Spectrom. 2003; 17(11):1203-1211; Pettus B J, et al., Rapid Commun Mass Spectrom. 2004; 18(5):577-583) can be used to measure DH-S1P, S1P and other sphingolipids in biological samples. This is currently the state of the art approach to measure DH-S1P and other sphingolipids. Okajima and colleagues (Okajima F, Nippon Yakurigaku Zasshi. 2001; 118(6):383-388; Murata N, et al., Anal Biochem. 2000; 282(1):115-120) have developed a quantitative radio-receptor assay, for measurement of S1P, which was based on the competition of S1P in the samples with the labeled S1P on the S1P receptor S1P1/Edg-1. H1a and colleagues have developed a method to quantify S1P by immobilized metal affinity chromatography (IMAC) and involving high-performance liquid chromatography (HPLC) coupled to a fluorescence detector (Lee Y M, et al., Prostaglandins & other lipid mediators. 2007; 84(3-4):154-162).

The LCMS-based approach can measure a wide range of sphingolipids from a single sample. Typically up to ˜20 sphingolipids can be measured in an ‘sphingoid bases assay’ offered through the MUSC Lipidomics Facility (http://hcc.musc.eduiresearch/sharedresources/lipidomics/lipidomicsanalyticcore.htm) which distinguishes sphingosine, dihydrosphingosine, sphingoid base-1 phosphates and various ceramide species. The data indicates that that DH-S1P, S1P and C24 ceramide are all inversely correlated with occurrence of IHD. All of these can be simultaneously measured by LCMS.

Through the application of innovative technologies such as liquid chromatography-tandem mass spectrometry (LC-MS-MS) (Pettus, B. J., et al., Rapid Commun Mass Spectrom 17: 1203-1211; Pettus, B. J., et al., Rapid Commun Mass Spectrom 18: 577-583). It is only now practical to perform high throughput quantification of plasma lipoprotein-associated S1P and other bioactive lysosphingolipid species in large numbers of blood samples from human subjects. Through use of innovative quantitative technologies including multiplex microbead array analysis and Electric Cell-substrate Impedance Sensing (Giaever, I., et al., Proc Natl Acad Sci USA 88: 7896-7900; Kataoka, N., et al., Proc Natl Acad Sci USA 99: 15638-15643) (ECIS), it is also possible to determine qualitative differences in the sphingolipid signaling capacity of lipoproteins including their ability to elicit endothelial cell phosphoprotein activation and influence endothelial barrier function.

The CCHS samples levels of sphingolipids including S1P and DH-S1P were evaluated in an HDL enriched fraction of serum. The fraction was prepared from serum by removing apolipoprotein-B containing lipoproteins by dextran-sulphate precipitation with beads (Polymedco, Chicago, Ill.). The inverse correlation between DH-S1P and IHD was not observed in the total serum. Similarly, S1P was not found to be inversely correlated with occurrence when total serum was analyzed.

### B. DEFINITIONS

### 1. A, An, The

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

### 2. Activity

As used herein, the term “activity” refers to a biological activity.

### 3. Assaying

Assaying, assay, or like terms refers to an analysis to determine a characteristic of a substance, such as a molecule or a cell, such as for example, the presence, absence, quantity, extent, kinetics or dynamics.

### 4. Body Fluid

The term “body fluid,” as used herein is intended to include body fluids that may be extracted, isolated or sampled including fluids naturally occurring in the body (for example, urine, stool, blood—whole serum or plasma—, spinal fluid, cerebrospinal fluid, ocular lens liquid, semen, synovial fluid, peritoneal fluid, pleural fluid, sputum, lymph fluid, saliva, amniotic fluid, pus, lavage fluid, sweat, bile, and tears, etc.). Body fluid is also intended to include an artificial solution of fluid that has been equilibrated with the blood (or otherwise mixed with a naturally occurring body fluid) and thus taken up considerable fluid and solutes from the body. For example, in certain embodiments peritoneal fluid may be considered a body fluid. Peritoneal fluid is, for example, fluid found in the peritoneal cavity of an individual, often due to insertion of peritoneal dialysis buffer into the peritoneal cavity.

### 5. Cardiovascular Disease

The term “cardiovascular disease” or the like term refers to diseases related to the heart and the blood vessels or the circulation, such as atherosclerosis, ischemic heart disease to or cerebrovascular disease such as coronary artery disease including angina pectoris and myocardial infarction, stroke, vascular heart disease and peripheral vascular disorders such as peripheral arterial disease and occlusive arterial diseases.

### 6. Cardioprotective

The term “cardioprotective” or the like term refers to a substance, material, composition, lipid or molecule (i.e. HDL-C) that can decrease a subject's risk for developing CVD or treat a subject having a CVD.

### 7. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

### 8. Combinations

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a cell is disclosed and discussed and a number of modifications that can be made to a number of molecules including the cell are discussed, each and every combination and permutation of the cell and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### 9. Compound

For the purposes of the present disclosure the terms “compound,” “analog,” and “composition of matter” can be used and stand equally well for the chemical entities described herein, including all enantiomeric forms, diastereomeric forms, salts, and the like, and the terms “compound,” “analog,” and “composition of matter” are used interchangeably throughout the present specification.

### 10. Comprise

Throughout the description and claims of this specification, the word “comprise” and variations of the word, such as “comprising” and “comprises,” means “including but not limited to,” and is not intended to exclude, for example, other additives, components, integers or steps.

### 11. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

### 12. Positive Control

A “positive control” or like terms is a control that shows that the conditions for data collection can lead to data collection.

### 13. Characterizing

Characterizing or like terms refers to gathering information about any property of a substance, such as a ligand, molecule, marker, or cell, such as obtaining a measurement for the ligand, molecule, marker, or cell.

### 14. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

### 15. Cardiovascular Disease Drug

A “cardiovascular disease drug” is a substance, material or molecule that can treat cardiovascular disease or treat risk factors of getting cardiovascular disease. For example a cardiovascular drug can be a beta blocker, an ace inhibitor, and/or a cholesterol lowering medication, such as a statin.

### 16. Decrease

A “decrease” can refer to any change that results in a smaller amount of a composition, compound or action, such as drug use. Thus, a “decrease” can refer to a reduction in levels, function, or activity. Also for example, a decrease can be a change in the amount of drug use such that the drug use can be less than previously observed. Another example can be a decrease in the side effects in subjects administered a combination composition compared to side effects in subjects administered each compositions alone.

### 17. Detect

Detect or like terms refer to an ability of the apparatus and methods of the disclosure to discover, measure or sense a substance or molecule.

### 18. HDL

HDL stands for “high density lipoprotein.” Increased HDL cholesterol levels are traditionally associated with a lower risk of cardiovascular disease. As described herein HDL is not always associated with lower risk of cardiovascular disease because of low levels of sphingolipids associated with the HDL.

### 19. Higher

The terms “higher,” “increases,” “elevates,” or “elevation” or variants of these terms, refer to increases above basal levels, e.g., as compared to a control. The terms “low,” “lower,” “reduces,” or “reduction” or variation of these terms, refer to decreases below basal levels, e.g., as compared to a control. For example, basal levels are normal in vivo levels prior to, or in the absence of, or addition of an agent such as an agonist or antagonist to activity.

### 20. Indicator

An “indicator” or like terms is a thing that indicates. Specifically, “an indicator for the mode of action of the molecule” means a thing, such as the concentration S1P, DH-S1P or C24; 1-ceramide in comparison with a control of S1P, DH-S1P or C24; 1-ceramide, that can be interpreted that the molecule has an influence on a system.

### 21. LDL

“LDL” stands for “low density lipoprotein”. Most of the cholesterol in the blood comes from LDL. Elevated LDL cholesterol levels is a major risk factor for CVD.

### 22. VLDL

“VLDL” stands for “very low density lipoprotein” and is composed mostly of cholesterol, with little protein. VLDL is often called “bad cholesterol” because it deposits cholesterol on the wails of arteries. Increased levels of VLDL are associated with cardiovascular disease.

### 23. Machine

“Machine” or the like terms refers to a mechanically, electrically or electronically operated device for performing at least one task. For example, a machine can be a computer; an analytical instrument, such as a mass-spectrometer (i.e. LCMS); device for collecting body fluids, such as a syringe.

### 24. Material

Material is the tangible part of something (chemical, biochemical, biological, or mixed) that goes into the makeup of a physical object.

### 25. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

### 26. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

### 27. Drug Candidate Molecule

A drug candidate molecule or like terms is a test molecule which is being tested for its ability to function as a drug or a pharmacophore. In certain situations, this molecule may be considered as a lead molecule.

### 28. Modulate

To modulate, or forms thereof, means increasing, decreasing, or maintaining a cellular activity mediated through a cellular target. It is understood that wherever one of these words is used it is also disclosed that it could be, for example, 1%, 5%, 10%, 20%, 50%, 100%, 500%, or 1000% increased from a control, or it could be, for example, 1%, 5%, 10%, 20%, 50%, or 100% decreased from a control.

### 29. Normalizing

Normalizing or like terms means, adjusting data, or a response, for example, to remove at least one common variable. For example, if two responses are generated, one for a control and molecule acting on the cell, normalizing would refer to the action of comparing the control to the response of the molecule, and removing the response due to the control only, such that the normalized response would represent the response due to the response of the molecule.

### 30. Optionally

“Optional” or “optionally” means that the subsequently described event or circumstance may or may not occur, and that the description includes instances where said event or circumstance occurs and instances where it does not.

### 31. Or

The word “or” or like terms as used herein means any one member of a particular list and also includes any combination of members of that list.

### 32. Prevent

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits cardiovascular disease is disclosed, then reduces and prevents cardiovascular are also disclosed.

### 33. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### 34. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

### 35. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces cardiovascular disease” means lowering the amount or risk of cardiovascular that takes place relative to a standard or a control.

### 36. Relevant Standard or Relevant Average Standard

The term “relevant standard” or “relevant average standard” or the like term refers to a standard that is directly associated with data, i.e. a measured level, of sphingolipids. For example, a relevant standard to the sphingolipid concentration in a subject with high HDL-C could be either the sphingolipid concentration of a group of subjects with high HDL-C, with CVD or the sphingolipid concentration of a group of subjects with high HDL-C without CVD. Thus, a relevant standard includes comparing subjects with high HDL-C to subjects also having high HDL-C.

### 37. Response

A response or like terms is any reaction to any stimulation.

### 38. Signaling Pathway(s)

A “defined pathway” or like terms is a path of a cell from receiving a signal (e.g., an exogenous ligand) to a cellular response (e.g., increased expression of a cellular target). The signaling pathway can be either relatively simple or quite complicated.

### 39. Standard

A “standard” or the like term refers to measured value or average value for a particular set of data. For example, a standard can be the average sphingolipid concentration for a group of subjects having high HDL-C and IHD or the average sphingolipid concentration for a group of subjects having high HDL-C without IHD.

### 40. Subject

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

### 41. Substance

A substance or like terms is any physical object. A material is a substance. Molecules, ligands, markers, cells, proteins, and DNA can be considered substances. A machine or an article would be considered to be made of substances, rather than considered a substance themselves.

### 42. Traditional Risk Factors of Having or Developing Cardiovascular Disease

The phrase “traditional risk factors of having or developing cardiovascular disease” or the like phrase refers to commonly accepted indicators of cardiovascular disease, such as high LDL-C, high VLDL-C, low HDL-C and high total cholesterol, known at the time of filing of this application.

### 43. Treatment

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

### 44. Therapeutic Effective

The term “therapeutically effective” means that the amount of the composition used is of sufficient quantity to ameliorate one or more causes or symptoms of a disease or disorder. Such amelioration only requires a reduction or alteration, not necessarily elimination. The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

### 45. Weight/%

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound. A weight percent of a component, unless specifically stated to the contrary, is based on the total weight of the formulation or composition in which the component is included.

### C. METHODS

Disclosed herein are tests, assays and screens that measure levels of DH-S1P and/or the ratio of [DH-S1P] μM/[apoAI] μM can be used as a indicator of occurrence or relative risk for cardiovascular disease such that low levels of DH-S1P or low ratios of [DH-S1P] μM/[apoAI] μM are indicative occurrence or of increased cardiovascular disease risk. Therefore, techniques that can be used clinically to measure DH-S1P (e.g., LCMS) in serum or in LDL/VLDL depleted serum samples (i.e., using rapid methods of isolating HDL such as dextran sulfate/magnesium chloride precipitation) should be performed.

Embodiments of the invention comprise therapies that act to increase plasma HDL-DH-S1P levels and decrease the risk for ischemic heart disease. For instance fortification of HDL memetics which are currently in clinical trials (see apoA-I mimetic peptide D-4F studies I http://www.jlr.org/cgi/content/abstract/49/6/1344) with DH-S1P.)

An additional embodiment is a diagnostic blood screen to measure DH-S1P levels in plasma HDL as a means of assessing relative risk of cardiovascular disease. For example, clinical LC-MS instruments that can measure DH-S1P and other sphingolipids in small volumes of blood. An additional embodiment is the use of DH-S1P is as an anti-inflammatory agent. ApoA-I mimetic peptides have been shown to be effect is a as anti-inflammatory agents. Their activity can be improved by fortification with DH-S1P.

DH-S1P has been shown to antagonize TGF-β signaling. Thus, DH-S1P-fortified HDL particles may be used to control pathological effects of TGF-β (e.g., accumulation of extracellular matrix in fibrosis or increased TGF-β signaling in aortic aneurysm) via modulation of the G protein-coupled receptor I signaling and new therapeutic approaches for treatment of fibrotic diseases.

Additional embodiments comprise methods of identifying, subjects, such as humans that are at risk of developing ischemic heart disease (IHD) or displaying IHD. Embodiments can be used to assist humans to take steps to either obviate this (i.e., therapeutically augment DH-S1P or S1P levels) or other factors that could contribute to increased risk through diet, exercise or drug therapy.

Therapeutic drugs that can be useful rectify pathologic lysosphingolipid peptides levels can comprise the administration of DH-S1P complexed to HDL or apolipoproteins (apoA-I, albumin) or apolipoprotein mimetics would be expected to alter HDL-associate DH-S1P levels and can effect disease.

Disclosed herein are methods of diagnosing cardiovascular disease in a subject, comprising the steps of:
a. collecting body fluid from the subject;
b. measuring the level of at least one sphingolipid in the body fluid;
c. diagnosing cardiovascular disease in a subject based on the measured level of sphingolipids.

Also disclosed herein are methods predicting cardiovascular disease in a subject, comprising the steps of:
a. collecting body fluid from the subject;
b. measuring the level of at least one sphingolipid in the body fluid;
c. predicting cardiovascular disease in a subject based on the measured level of sphingolipids.

Also disclosed herein are methods of identifying a subject at risk of developing cardiovascular disease in a subject, comprising the steps of:
a. collecting body fluid from the subject;
b. measuring the level of at least one sphingolipid in the body fluid;
c. identifying a subject at risk of developing cardiovascular disease based on the measured level of sphingolipids.

Also disclosed here in is a method of treating cardiovascular disease in a subject, comprising administering a composition elevating sphingolipid levels in a subject.

Also disclosed herein is a method of treating cardiovascular disease in a subject comprising administering a cardiovascular disease drug, such as a beta blocker, an ace inhibitor, and/or a cholesterol lowering medication, such as a statin.

In some forms of the methods one or more steps can be performed by a machine. For example step a., b., or c could each individually be performed by a machine. In one embodiment at least step b. is performed by a machine. The machine can be any type of machine able to perform the desired task. The machine can be, for example, a syringe. computer or a mass spectrometer. In one embodiment a machine is necessary to perform the methods. For example, collecting body fluid can be done with a syringe. In one embodiment a LCMS can measure at least one sphingolipid in the body fluid. In one embodiment the LCMS simultaneously measures multiple sphingolipid in the body fluid. In one embodiment a machine is necessary to perform the methods.

In some forms a LCMS can measure multiple sphinolipids in the body fluid. In one embodiment the LCMS can measure at least 3, 5, 7, 9, 11, 13, 15, 17, 19 or 21 sphingoplipids. In one embodiment the LCMS can measure at least 3, 5, 7 or 9, sphingoplipids. In one embodiment the LCMS can measure at least 3 sphingolipids. The measured sphingolipids can be predetermined before the measurement. In one embodiment the sphinolipids can be S1P, DH-S1P or C24:1-ceramide.

In some forms of the methods the sphingolipid can be associated with another substance or molecules. In one embodiment the sphinolipids can be associated with cholesterol. In one embodiment the sphinolipids can be associated HDL-C or albumin.

In some forms of the methods the body fluid can be blood or plasma. In one embodiment the body fluid can be plasma. In another embodiment the body fluid can be blood. In another embodiment the body fluid is manipulated before it is measured for sphingolipids. The body fluid can be manipulated by removing unwanted substances from the body fluid. In one embodiment LDL and VLDL are substantially removed from the body fluid.

In some forms of the methods diagnosing cardiovascular disease comprises comparing the measured level of sphingolipids to a standard. The standard can be, for example, a measured average value from a particular set of data. In one embodiment the standard can be the sphingolipid concentration of subjects with high HDL-C. In another embodiment the standard can be the sphingolipid concentration of subjects with low HDL-C. Comparing a measured level of sphingolipids to a standard can be done by electing a standard that is relevant to the measured value. For example, the sphingolipid concentration from a subject having high HDL-C can be compared to a standard derived from subject having high HDL-C. The standard can be derived from subject with or without cardiovascular disease. The standard can be derived using different particular sets of data defining the standard. For example, the particular set of data can be age, gender, HDL-C level, cardiovascular disease, not having cardiovascular disease.

The sphingolipid can be any sphingolipid. In one embodiment the sphingolipid can be associated with cholesterol. In another embodiment the sphingolipid can be S1P, DH-S1P or C24:1-ceramide.

In some forms of the methods a subject can be diagnosed with cardiovascular disease when the measured level of at least one sphingolipids is at least 10%, 15%, 20%, 25%, 30%, 35%, 40%, 45% or 50% lower than a relevant average standard level of sphingolipids in subjects without cardiovascular disease. In one embodiment a subject can be diagnosed with cardiovascular disease when the measured level of at least one sphingolipids is at least 25%, 30%, 35%, 40%, 45% or 50% lower than a relevant average standard level of sphingolipids in subjects without cardiovascular disease. In another embodiment a subject can be diagnosed with cardiovascular disease when the measured level of at least one sphingolipids is at least 35%, 40%, 45% or 50% lower than a relevant average standard level of sphingolipids in subjects without cardiovascular disease.

In some forms of the methods a subject can be diagnosed with cardiovascular disease when the measured level of S1P is at least 0.3 μM, 0.4 μM, 0.5 μM, 0.6 μM, 0.7 μM, 0.8 μM, 0.9 μM or 1.0 μM lower than a relevant average standard level of S1P in subjects without cardiovascular disease. In one embodiment a subject can be diagnosed with cardiovascular disease when the measured level of S1P is at least 0.6 μM, 0.7 μM, 0.8 μM, 0.9 μM or 1.0 μM lower than a relevant average standard level of S1P in subjects without cardiovascular disease. In one embodiment a subject can be diagnosed with cardiovascular disease when the measured level of S1P is at least 0.8 μM, 0.9 μM or 1.0 μM lower than a relevant average standard level of S1P in subjects without cardiovascular disease. In some forms of the methods a subject can be diagnosed with cardiovascular disease when the measured level of DH-S1P is at least 0.04 μM, 0.06 μM, 0.08 μM, 0.1 μM, 1.2 μM, 1.4 μM, 1.6 μM, 1.8 μM or 2.0 μM lower than a relevant average standard level of DH-S1P in subjects without cardiovascular disease. In one embodiment a subject can be diagnosed with cardiovascular disease when the measured level of DH-S1P is at least 0.1 μM, 1.2 μM, 1.4 μM, 1.6 μM, 1.8 μM or 2.0 μM lower than a relevant average standard level of DH-S1P in subjects without cardiovascular disease. In another embodiment a subject can be diagnosed with cardiovascular disease when the measured level of DH-S1P is at least 1.6 μM, 1.8 μM or 2.0 μM lower than a relevant average standard level of DH-S1P in subjects without cardiovascular disease.

In some forms of the methods a subject can be diagnosed with cardiovascular disease when the measured level of C24:1-ceramide is at least 0.04 μM, 0.05 μM, 0.06 μM, 0.07 μM, 0.08 μM, 0.09 μM, or 0.1 μM lower than a relevant average standard level of C24:1-ceramide in subjects without cardiovascular disease. In one embodiment a subject can be diagnosed with cardiovascular disease when the measured level of C24:1-ceramide is at least 0.07 μM, 0.08 μM, 0.09 μM, or 0.1 μM lower than a relevant average standard level of C24:1-ceramide in subjects without cardiovascular disease. In another embodiment a subject can be diagnosed with cardiovascular disease when the measured level of C24:1-ceramide is at least 0.09 μM, or 0.1 μM lower than a relevant average standard level of C24:1-ceramide in subjects without cardiovascular disease.

In some forms of the methods a subject can be diagnosed with cardiovascular disease when the [S1P] μM/[apoAI] μM ratio is at least 0.005, 0.007, 0.009, 0.011 or 0.013 lower than a relevant average standard level of [S1P] μM/[apoAI] μM ratio in subjects without cardiovascular disease. In one embodiment a subject can be diagnosed with cardiovascular disease when the [DH-S1P] μM/[apoAI] μM ratio is at least 0.0009, 0.0011 or 0.0013 lower than a relevant average standard level of [DH-S1P] μM/[apoAI] μM ratio in subjects without cardiovascular disease. In another embodiment a subject can be diagnosed with cardiovascular disease when the [DH-S1P] μM/[apoAI] μM ratio is at least 0.0005, 0.0007, 0.0009, 0.0011 or 0.0013 lower than a relevant average standard level of [DH-S1P] μM/[apoAI] μM ratio in subjects without cardiovascular disease.

In some forms of the methods a subject can be diagnosed with cardiovascular disease the [C24:1-ceramide] μM/[apoAI] μM ratio is at least 0.0005, 0.0007, 0.0009, 0.0011 or 0.0013 lower than a relevant average standard level of [C24:1-ceramide] μM/[apoAI] μM ratio in subjects without cardiovascular disease.

In some forms a subject can be diagnosed with cardiovascular disease when the measured level of at least one sphingolipids is statistically lower than a relevant average standard level of sphingolipids in subjects without cardiovascular disease. For example, the statistical analysis can be performed using ANOVA. The sphingolipid can be determined to be statistically lower if it in a p-test has a p value of <0.05.

In some forms of the methods a subject can be predicted to have cardiovascular disease when the measured level of at least one sphingolipids is at least 10%, 15%, 20%, 25%, 30%, 35%, 40%, 45% or 50% lower than a relevant average standard level of sphingolipids in subjects without cardiovascular disease. In one embodiment a subject can be predicted to have cardiovascular disease when the measured level of at least one sphingolipids is at least 25%, 30%, 35%, 40%, 45% or 50% lower than a relevant average standard level of sphingolipids in subjects without cardiovascular disease. In another embodiment a subject can be predicted to have cardiovascular disease when the measured level of at least one sphingolipids is at least 35%, 40%, 45% or 50% lower than a relevant average standard level of sphingolipids in subjects without cardiovascular disease.

In some forms of the methods a subject can be predicted to have cardiovascular disease when the measured level of S1P is at least 0.3 μM, 0.4 μM, 0.5 μM, 0.6 μM, 0.7 μM, 0.8 μM, 0.9 μM or 1.0 μM lower than a relevant average standard level of S1P in subjects without cardiovascular disease. In one embodiment a subject can be predicted to have cardiovascular disease when the measured level of S1P is at least 0.6 μM, 0.7 μM, 0.8 μM, 0.9 μM or 1.0 μM lower than a relevant average standard level of S1P in subjects without cardiovascular disease. In one embodiment a subject can be predicted to have cardiovascular disease when the measured level of S1P is at least 0.8 μM, 0.9 μM or 1.0 μM lower than a relevant average standard level of S1P in subjects without cardiovascular disease. In some forms of the methods a subject can be predicted to have cardiovascular disease when the measured level of DH-S1P is at least 0.04 μM, 0.06 μM, 0.08 μM, 0.1 μM, 1.2 μM, 1.4 μM, 1.6 μM, 1.8 μM or 2.0 μM lower than a relevant average standard level of DH-S1P in subjects without cardiovascular disease. In one embodiment a subject can be predicted to have cardiovascular disease when the measured level of DH-S1P is at least 0.1 μM, 1.2 μM, 1.4 μM, 1.6 μM, 1.8 μM or 2.0 μM lower than a relevant average standard level of DH-S1P in subjects without cardiovascular disease. In another embodiment a subject can be predicted to have cardiovascular disease when the measured level of DH-S1P is at least 1.6 μM, 1.8 μM or 2.0 μM lower than a relevant average standard level of DH-S1P in subjects without cardiovascular disease.

In some forms of the methods a subject can be predicted to have cardiovascular disease when the measured level of C24:1-ceramide is at least 0.04 μM, 0.05 μM, 0.06 μM, 0.07 μM, 0.08 μM, 0.09 μM, or 0.1 μM lower than a relevant average standard level of C24:1-ceramide in subjects without cardiovascular disease. In one embodiment a subject can be predicted to have cardiovascular disease when the measured level of C24:1-ceramide is at least 0.07 μM, 0.08 μM, 0.09 μM, or 0.1 μM lower than a relevant average standard level of C24:1-ceramide in subjects without cardiovascular disease. In another embodiment a subject can be predicted to have cardiovascular disease when the measured level of C24:1-ceramide is at least 0.09 μM, or 0.1 μM lower than a relevant average standard level of C24:1-ceramide in subjects without cardiovascular disease.

In some forms of the methods a subject can be predicted to have cardiovascular disease when the [S1P] μM/[apoAI] μM ratio is at least 0.005, 0.007, 0.009, 0.011 or 0.013 lower than a relevant average standard level of [S1P] μM/[apoAI] μM ratio in subjects without cardiovascular disease. In one embodiment a subject can be predicted to have cardiovascular disease when the [DH-S1P] μM/[apoAI] μM ratio is at least 0.0009, 0.0011 or 0.0013 lower than a relevant average standard level of [DH-S1P] μM/[apoAI] μM ratio in subjects without cardiovascular disease. In another embodiment a subject can be predicted to have cardiovascular disease when the [DH-S1P] μM/[apoAI] μM ratio is at least 0.0005, 0.0007, 0.0009, 0.0011 or 0.0013 lower than a relevant average standard level of [DH-S1P] μM/[apoAI] μM ratio in subjects without cardiovascular disease.

In some forms of the methods a subject can be predicted to have cardiovascular disease the [C24:1-ceramide] μM/[apoAI] μM ratio is at least 0.0005, 0.0007, 0.0009, 0.0011 or 0.0013 lower than a relevant average standard level of [C24:1-ceramide] μM/[apoAI] μM ratio in subjects without cardiovascular disease.

In some forms a subject can be predicted to have cardiovascular disease when the measured level of at least one sphingolipids is statistically lower than a relevant average standard level of sphingolipids in subjects without cardiovascular disease. For example, the statistical analysis can be performed using ANOVA. The sphingolipid can be determined to be statistically lower if it in a p-test has a p value of <0.05 relevant to the standard.

In some forms of the methods a subject can be identified to be at risk of developing cardiovascular disease when the measured level of at least one sphingolipids is at least 10%, 15%, 20%, 25%, 30%, 35%, 40%, 45% or 50% lower than a relevant average standard level of sphingolipids in subjects without cardiovascular disease. In one embodiment a subject can be identified to be at risk of developing cardiovascular disease when the measured level of at least one sphingolipids is at least 25%, 30%, 35%, 40%, 45% or 50% lower than a relevant average standard level of sphingolipids in subjects without cardiovascular disease. In another embodiment a subject can be identified to be at risk of developing cardiovascular disease when the measured level of at least one sphingolipids is at least 35%, 40%, 45% or 50% lower than a relevant average standard level of sphingolipids in subjects without cardiovascular disease.

In some forms of the methods a subject can be identified to be at risk of developing cardiovascular disease when the measured level of S1P is at least 0.3 μM, 0.4 μM, 0.5 μM, 0.6 μM, 0.7 μM, 0.8 μM, 0.9 μM or 1.0 μM lower than a relevant average standard level of S1P in subjects without cardiovascular disease. In one embodiment a subject can be identified to be at risk of developing cardiovascular disease when the measured level of S1P is at least 0.6 μM, 0.7 μM, 0.8 μM, 0.9 μM or 1.0 μM lower than a relevant average standard level of S1P in subjects without cardiovascular disease. In one embodiment a subject can be identified to be at risk of developing cardiovascular disease when the measured level of S1P is at least 0.8 μM, 0.9 μM or 1.0 μM lower than a relevant average standard level of S1P in subjects without cardiovascular disease. In some forms of the methods a subject can be identified to be at risk of developing cardiovascular disease when the measured level of DH-S1P is at least 0.04 μM, 0.06 μM, 0.08 μM, 0.1 μM, 1.2 μM, 1.4 μM, 1.6 μM, 1.8 μM or 2.0 μM lower than a relevant average standard level of DH-S1P in subjects without cardiovascular disease. In one embodiment a subject can be identified to be at risk of developing cardiovascular disease when the measured level of DH-S1P is at least 0.1 μM, 1.2 μM, 1.4 μM, 1.6 μM, 1.8 μM or 2.0 μM lower than a relevant average standard level of DH-S1P in subjects without cardiovascular disease. In another embodiment a subject can be identified to be at risk of developing cardiovascular disease when the measured level of DH-S1P is at least 1.6 μM, 1.8 μM or 2.0 μM lower than a relevant average standard level of DH-S1P in subjects without cardiovascular disease.

In some forms of the methods a subject can be identified to be at risk of developing cardiovascular disease when the measured level of C24:1-ceramide is at least 0.04 μM, 0.05 μM, 0.06 μM, 0.07 μM, 0.08 μM, 0.09 μM, or 0.1 μM lower than a relevant average standard level of C24:1-ceramide in subjects without cardiovascular disease. In one embodiment a subject can be identified to be at risk of developing cardiovascular disease when the measured level of C24:1-ceramide is at least 0.07 μM, 0.08 μM, 0.09 μM, or 0.1 μM lower than a relevant average standard level of C24:1-ceramide in subjects without cardiovascular disease. In another embodiment a subject can be identified to be at risk of developing cardiovascular disease when the measured level of C24:1-ceramide is at least 0.09 μM, or 0.1 μM lower than a relevant average standard level of C24:1-ceramide in subjects without cardiovascular disease.

In some forms of the methods a subject can be identified to be at risk of developing cardiovascular disease when the [S1P] μM/[apoAI] μM ratio is at least 0.005, 0.007, 0.009, 0.011 or 0.013 lower than a relevant average standard level of [S1P] μM/[apoAI] μM ratio in subjects without cardiovascular disease. In one embodiment a can be identified to be at risk of developing cardiovascular disease when the [DH-S1P] μM/[apoAI] μM ratio is at least 0.0009, 0.0011 or 0.0013 lower than a relevant average standard level of [DH-S1P] μM/[apoAI] μM ratio in subjects without cardiovascular disease. In another embodiment a subject can be identified to be at risk of developing cardiovascular disease when the [DH-S1P] μM/[apoAI] μM ratio is at least 0.0005, 0.0007, 0.0009, 0.0011 or 0.0013 lower than a relevant average standard level of [DH-S1P] μM/[apoAI] μM ratio in subjects without cardiovascular disease.

In some forms of the methods a subject can be identified to be at risk of developing cardiovascular disease the [C24:1-ceramide] μM/[apoAI] μM ratio is at least 0.0005, 0.0007, 0.0009, 0.0011 or 0.0013 lower than a relevant average standard level of [C24:1-ceramide] μM/[apoAI] μM ratio in subjects without cardiovascular disease.

In some forms a subject can be identified to be at risk of developing cardiovascular disease when the measured level of at least one sphingolipids is statistically lower than a relevant average standard level of sphingolipids in subjects without cardiovascular disease. For example, the statistical analysis can be performed using ANOVA. The sphingolipid can be determined to be statistically lower if it in a p-test has a p value of <0.05 relevant to the standard.

In some forms of the methods the cardiovascular disease can be ischemic heart disease. In one embodiment the ischemic heart disease can be arthrosclerosis.

In some forms of the methods the subject could have no traditional risk factors of having or developing cardiovascular disease. In some forms of the methods the subject does not have conventional risk factors associated with cardiovascular disease. In one embodiment the conventional risk factor can be elevated LDL-C or low HDL-C.

In one embodiment the methods can further comprise reducing LDL-C level from the body fluid.

In some forms of the methods the subject can have high HDL-C.

In some forms of the methods the subject is in need of treatment for cardiovascular disease. For example, the subject can have or be at risk of having cardiovascular disease.

In some forms of the methods the subject is monitored for cardiovascular disease. For example, the subject can routinely get tested for cardiovascular disease or sign of cardiovascular disease. The subject can also be monitored both before and after being diagnosed or identified as a subject in risk of having cardiovascular disease.

In some forms of the methods the subject diagnosed with cardiovascular disease

Also disclosed here in is a method of treating cardiovascular disease in a subject, comprising administering a composition elevating sphingolipid levels in a subject.

In some forms of the methods the sphingolipid level can be S1P, DH-S1P or C24 ceramide.

In some forms of the methods the composition can comprise S1P, DH-S1P or C24 ceramide.

In some forms of the methods the composition can comprise a statin.

### D. EXAMPLES

### 1. Example 1

### Sphingolipids and IHD

i. Material and Methods

Study group: The study involved the analysis of blood serum samples existing in the Copenhagen City Heart Study collection (CCHS)). (Schnohr, P., et al., Ugeskrift for laeger 139: 1921-1923; Schnohr, P., et al., European heart journal 23: 620-626). The CCHS is a prospective cardiopulmonary study of 20- to 93-year-old Danes of both sexes sampled from the general population in 1976-1978 and reexamined in 1981-1983, 1991-1994 and 2001-2003. (Juul, K., et al., Blood 100: 3-10) Informed consent was obtained from all participants. The Ethics Committee of Copenhagen and Frederiksberg approved the study (study No. 100.2039/91). Based on analysis of the total population (individuals from CCHS without ischemic heart disease (IHD), n=5,911 [women=3,384; men=2,527]) the average normal level of HDL-C for women is 62.1±18.9 mg/dL and 50.7±16.4 mg/dL for men. Four groups of CCHS samples were selected for testing which included 55 samples from individuals with high HDL-C (80.3±14.3 mg/dL) and no evidence of IHD, 53 samples from gender and age matched individuals with high HDL-C (78.8±14.2 mg/dL) and verified IHD, 54 samples from individuals with low HDL-C (33.7±5.8 mg/dL) and no evidence of IHD and 42 samples from gender and age matched individuals with low HDL-C (31.8±5.3 mg/dL) and verified IHD. All individuals had LDL-C<160 mg/dL, triglycerides<150 mg/dL, and none were treated with LDL-C-lowering medications. Subjects had an average age of 62 years, approximately 30% were women and 8.5% had diabetes mellitus. The characteristics of subjects from which samples were derived are summarized in Table 1.

Sphingolipid analysis of CCHS serum samples: Blinded liquid chromatography-mass spectrometry (LC-MS-MS) sphingolipid analysis was performed on aliquots of LDL- and VLDL-depleted, HDL-containing preparations made from CCHS serum samples. To prepare LDL- and VLDL-depleted, HDL-containing preparations, CCHS serum samples were subjected to magnetic bead-dextran-sulfate/MgCl₂ precipitation (Reference Diagnostics, Inc. Bedford, Mass.) to remove apolipoprotein B (apoB)-containing particles (i.e., LDL and VLDL) (Warnick, G. R., et al., Clin Chem 28: 1379-1388). Total cholesterol levels in the supernatants were measured by an enzymatic method using a commercially available kit (Wako Pure Chemical Co., Osaka, Japan). Apolipoprotein A-I (apoA-I) levels were quantified enzymatically in HDL-containing preparations on a Cobas Fara analyzer (Roche Diagnostics Systems, Inc) using Sigma reagents.

Aliquots of the supernatants were subjected to LC-MS-MS analysis on a Thermo Finnigan TSQ 7000 triple quadrupole mass spectrometer, operating in a Multiple Reaction Monitoring (MRM) positive ionization mode, using modified version of the protocol described by Bielawski et al. (Methods 39: 82-91). Briefly, CCHS samples (50 μl diluted 1:2 with Dulbecco's PBS) were fortified with the internal standards (ISs: C17 base D-erythro-sphingosine (17CSph), C17 S1P (17CS1P), N-palmitoyl-D-erythro-C13 sphingosine (13C16-Cer) and heptadecanoyl-D-erythro-sphingosine (C17-Cer)), and extracted with ethyl acetate/iso-propanol/water (60/30/10 v/v) solvent system. After evaporation and reconstitution in 100 μl of methanol, samples were injected on the HP1100/TSQ 7000 LC/MS system and gradient eluted from the BDS Hypersil C8, 150×3.2 mm, 3 μm particle size column, with 1.0 mM methanolic ammonium formate/2 mM aqueous ammonium formate mobile phase system. Peaks corresponding to the target analytes and internal standards were collected and processed using the Xcalibur software system. Quantitative analysis was based on the calibration curves generated by spiking an artificial matrix with the known amounts of the target analyte synthetic standards and an equal amount of the internal standards (ISs). The target analyte/IS peak areas ratios were plotted against analyte concentration. The target analyte/IS peak area ratios from the samples were similarly normalized to their respective ISs and compared to the calibration curves, using a linear regression model.

Statistical analysis of data: Pairwise comparisons were performed between the groups of LC-MS-MS data and Tukey's adjustment for multiple comparisons was used to control the Type I error rate associated with the pairwise comparisons. The one-way ANOVA model was fit using proc glm in SAS v9.1.3. The Studentized residuals were calculated and assessed for the model assumptions of normality and constant variance. It was further assumed that the samples are independent of one another. Hypothesis tests were conducted at level of significance 0.05. Data were presented as box plots using the KaleidaGraph (version 4.0, Synergy Software, Reading, Pa.).

ii. Results

Inverse correlation of HDL-associated S1P, DH-S1P and C24:1-ceramide with ischemic heart disease: CCHS subjects were categorized into four groups based on having high or low HDL-C and the presence or absence of IHD (Table 1). Serum from each subject was subjected to dextran sulfate/MgCl₂ precipitation to prepare a LDL- and VLDL-depleted, HDL-containing fraction. On average, total cholesterol levels in these HDL preparations were found to be within 10% of the HDL-C levels measured in total serum. LC-MS-MS sphingolipid composition analysis was performed on the LDL- and VLDL-depleted, HDL-containing fractions of serum. The results of LC-MS-MS analysis are summarized in Table 2. The major sphingolipids associated with the HDL-containing fractions were S1P, DH-S1P, C24:1 ceramide and C24 ceramide. Statistical analysis of the LC-MS-MS data showed that S1P and DH-S1P levels were significantly lower (p<0.0001) in the HDL-containing serum fractions from individuals with high HDL-C having IHD as compared to individuals with high HDL-C having no evidence of IHD (FIGS. 1A and B). Furthermore, S1P and DH-S1P levels were significantly lower (p<0.0001) in HDL-containing fractions from individuals with low HDL-C and having IHD as compared to individuals with low HDL-C having no evidence of IHD (FIGS. 1A and B).

Among the other sphingolipids analyzed in the HDL-containing fractions from the CCHS subjects, C24:1-ceramide levels were significantly lower (p=0.006) in HDL-containing fractions from individuals with high HDL-C having IHD as compared to individuals with high HDL-C having no evidence of IHD (FIG. 1C). Furthermore, C24:1-ceramide levels were significantly lower (p=0.0007) in samples from individuals with low HDL-C and having IHD as compared to individuals with low HDL-C having no evidence of IHD (FIG. 1C).

When S1P, DH-S1P and C24:1-ceramide levels were assessed relative to the concentration of apoA-I in the samples, the ratio of the concentration of these sphingolipids to apoA-I concentration were all significantly lower (p<0.05) in samples from individuals with high HDL-C having IHD as compared to individuals with high HDL-C having no evidence of IHD (FIG. 2A-C). Furthermore, these ratios were also significantly lower (p<0.05) in samples from individuals with low HDL-C and having IHD as compared to individuals with low HDL-C having no evidence of IHD (FIG. 2A-C).

iii. Discussion

This study was undertaken to test whether compositional differences in sphingolipid content of HDL is important in deciphering the putative protective role of HDL in IHD. The findings indicate that levels of S1P, DH-S1P and C24:1-ceramide in the HDL-containing fraction of serum inversely correlate with the occurrence of IHD. This inverse correlation applied to HDL isolated from subjects with IHD regardless of their having high or low HDL-C levels.

While there is much known about that bioactivities of S1P, particularly in the context of vascular biology, little is known as to the biological actions of DH-S1P and C24:1-ceramide. Recently, DH-S1P has been shown to mediate activation ERK1/2 and induction of matrix metalloproteinase 1 (MMP1) expression in dermal fibroblasts (Bu, S., et al., Faseb J 20: 184-186). These effects were not reproduced by S1P and the receptor responsible is yet to be identified. MMP1 is believed to play an important role in the pathogenesis of atherosclerosis. Findings from mouse studies indicates that MMP1 can inhibit atherosclerosis (Lemaitre, V., et al., J Clin Invest 107: 1227-1234) and recent human studies show that persons homozygous for a transcriptionally overactive allele of the MMP1 have a reduced risk of coronary heart disease (Ye, S., et al., European heart journal 24:1668-1671). Thus, lower levels of HDL-associated DH-S1P could be predicted to reduce MMP1 expression and consequently its atheroprotective effects.

Blood levels of S1P, DH-S1P and C24:1-ceramide and/or the ratio of the concentrations of these sphingolipids normalized to serum apolipoprotein A-I levels can be clinically useful to assess relative risk for cardiovascular disease such that low levels of S1P or DH-S1P or low ratios of either [S1P] μM/[apoAI] μM or [DH-S1P] μM/[apoAI] μM can be indicative of increased cardiovascular disease risk. Also therapies that act to increase plasma HDL-S1P levels could decrease the risk for IHD.

### 2. Example 2

### Dose Dependant Relationship Between S1P, DH-S1P and IDH

i. Experimental Results

a. Data Description:

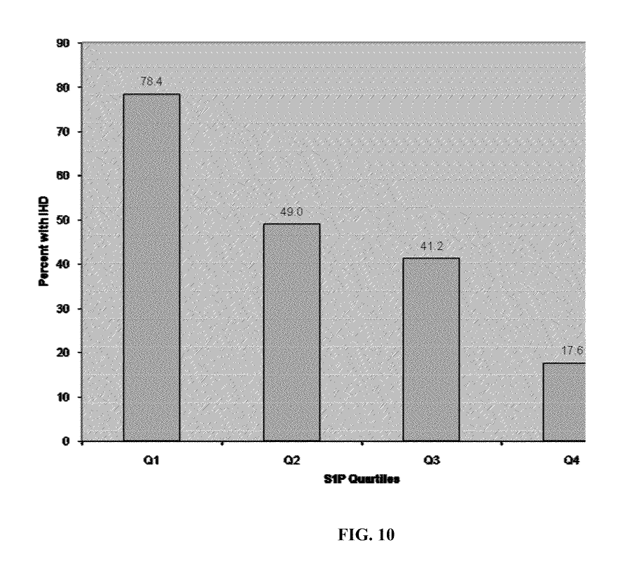

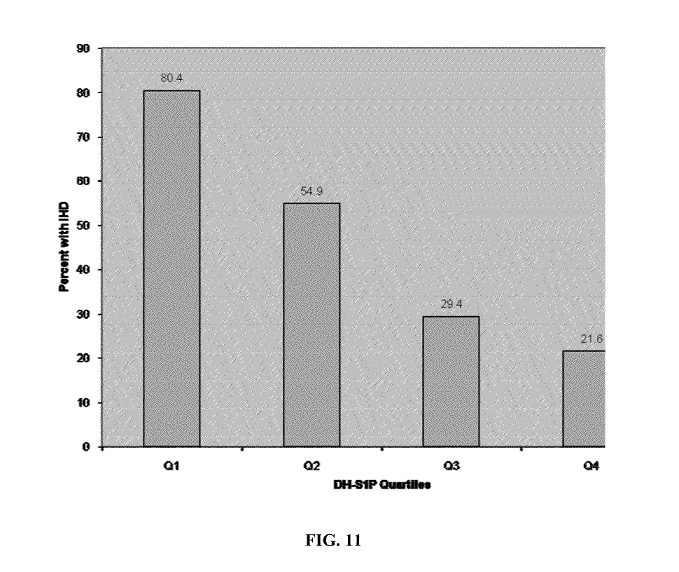

Two hundred and four samples were obtained from the Copenhagen City Heart Study (CCHS). These included 55 samples from individuals with high HDL and no evidence of ischemic heart disease (IHD), 53 samples from individuals with high HDL and evidence of IHD, 54 samples from individuals with low HDL and no evidence of IHD, and 42 samples from individuals with low HDL and evidence of IHD. Blinded liquid chromatography/mass spectrometry sphingolipid analysis was performed on the samples. Graphical descriptives of the data are presented in Table 4 and 5 and FIGS. 10 and 11.

FIG. 10 show that categorizing the S1P level inevitably results in a loss of information; however, categorizing the data in this way can be used to demonstrate whether a dose-dependent relationship between S1P and IHD exists. The quartiles (Q1 2.34173, Q2 3.01517, Q3 3.78407 μM) as an objective way of defining the categories. In this case, the traditional χ2 test can be used to test for an association. The Cochran-Armitage test was also conducted for linear trend. The result was statistically significant (Z=6.0887, p-value<0.0001), indicating that the proportion of subjects with IHD decreases as S1P (as defined by the corresponding quartile) increases.

FIG. 11 shows that categorizing the DH-S1P level inevitably results in a loss of information; however, categorizing the data in this way can be used to demonstrate whether a dose-dependent relationship between DH-S1P and IHD exists. We use the quartiles (Q1 0.20023340, Q2 0.30054029, Q3 0.39324481 μM) as an objective way of defining the categories. In this case, the traditional χ2 test can be used to test for an association. The Cochran-Armitage test was also conducted for linear trend. The result was statistically significant (Z=6.4654, p-value<0.0001), indicating that the proportion of subjects with IHD decreases as DH-S1P (as defined by the corresponding quartile) increases.

The primary purpose of this analysis is to demonstrate that the proportion of subjects with IHD tends to decrease as S1P levels increase. While we could approach this using S1P as a continuous variable, we are interested in defining cutpoints of S1P that describe an increased proportion of subjects with IHD. As a result, we begin by grouping subjects according to the corresponding quartile of S1P. The analysis was repeated using quartiles of DH-S1P.

TABLE 4
S1P Quartile by IHD Status

IHD

| Quartile (uM) | Negative | Positive | Total |
|---|---|---|---|
| 1 (S1P ≦ 2.34173) | 11 (21.57) | 40 (78.43) | 51 |
| 2 (2.34173 < S1P ≦ 3.01517) | 26 (50.98) | 25 (49.02) | 51 |
| 3 (3.01517 < S1P ≦ 3.78407) | 30 (58.82) | 21 (41.18) | 51 |
| 4 (S1P > 3.78407) | 42 (82.35) | 9 (17.65) | 51 |
| Total | 109 | 95 | 204 |

Frequency Missing = 1
Table 4 shows that an increase is S1P gives a decrease in IHD in patients in a study of 51 patients per group.

TABLE 5
DH-S1P Quartile by IHD Status

| | IHD | | |
|---|---|---|---|
| Quartile (uM) | Negative | Positive | Total |
| 1 (DH-S1P ≦ 0.20023340) | 10 (19.61) | 41 (80.39) | 51 |
| 2 (0.20023340 < DH-S1P ≦ 0.30054029) | 23 (45.10) | 28 (54.90) | 51 |
| 3 (0.30054029 < DH-S1P ≦ | 36 (70.59) | 15 (29.41) | 51 |
| 30.39324481) | | | |
| 4 (DH-S1P > 0.39324481) | 40 (78.43) | 11 (21.57) | 51 |
| Total | 109 | 95 | 204 |

Frequency Missing = 1
Table 5 shows that an increase is DH-S1P gives a decrease in IHD in patients in a study of 51 patients per group.

### 3. Example 3

### S1P Signaling

The study is focused on defining the role of S1P signaling on various aspects of vascular biology including the process of vasculogenesis (Argraves, K. M., et al., J Biol Chem 279: 50580-50590) and endothelial barrier activity, (Argraves, K. M., et al., J Biol Chem 283: 25074-25081), a major physiological function of the endothelium. Endothelial cell barrier dysfunction results in increased vascular permeability observed in inflammation, tumor angiogenesis, and atherosclerosis. In cultured endothelial cells, S1P acts to increase endothelial barrier activity as indicated by increased transendothelial electrical resistance (TEER) (Schaphorst, K. L., et al., Am J Physiol Lung Cell Mol Physiol 285: L258-267; Xu, M., et al., Am J Physiol Cell Physiol). Moreover, S1P administration greatly reduces lung capillary leakage induced in mice by lipopolysaccharide treatment (Peng, X., American journal of respiratory and critical care medicine 169: 1245-1251).

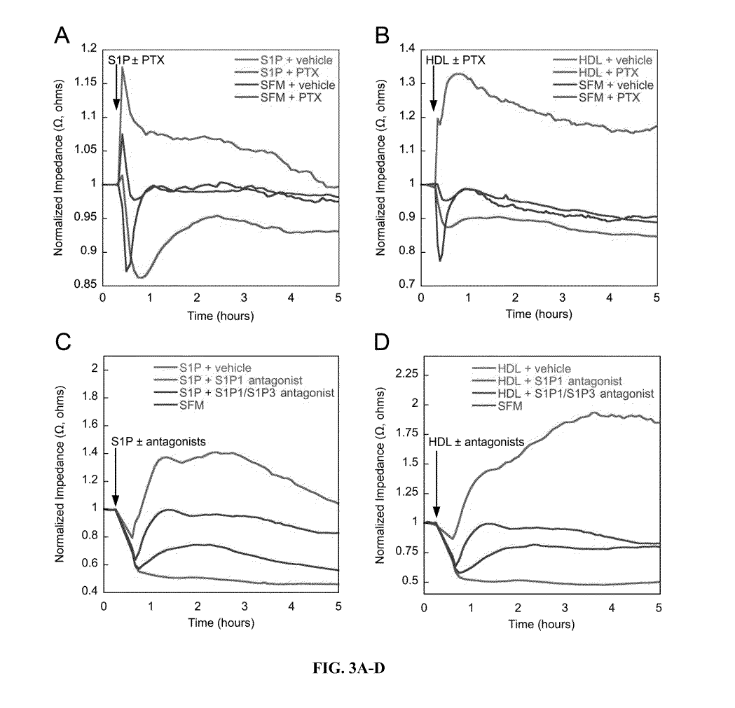

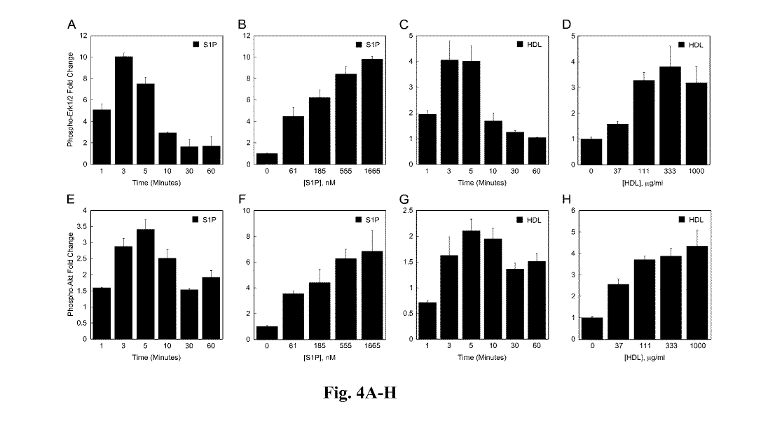

HDL-associated S1P promotes endothelial barrier function: Considering the fact that HDL is a physiological carrier of S1P, Electrical Cell Substrate Impedance Sensing (ECIS) Assay (Finnegan, J. H., et al., J Biol Chem 280: 17286-17293; Garcia, J. G., et al., J Clin Invest 108: 689-701) was used to investigate the possibility that HDL regulates endothelial barrier integrity in an S1P-dependent manner, (Argraves, K. M., et al., J Biol Chem 283: 25074-25081). As shown in FIG. 8A, S1P induces a dose-dependent increase in TEER. The TEER response to S1P displays a bimodal distribution over a 5 h period. HDL treatment also produces a dose dependent increase in TEER (see FIG. 8B). Like S1P, HDL also produced a bimodal TEER response. To implicate S1P signaling in the process of HDL-induced enhancement of TEER, the effects of pertussis toxin (PTX) was tested on the process since it inhibits Gi protein-coupled S1P receptor signaling in endothelial cells (Lee, M. J., et al., J Biol Chem 271: 11272-11279). As shown in FIG. 3A, the TEER response of endothelial cell monolayers to S1P treatments was inhibited by PTX. Similarly, the TEER response of HDL was completely abrogated by PTX (FIG. 3B), suggesting the requirement for Gi-coupled S1P receptors.

S1P receptor antagonists inhibit HDL-induced enhancement of TEER. The S1P receptor, S1P1, has been shown to mediate S1P stimulated augmentation of endothelial barrier activity (Garcia, J. G., et al., J Clin Invest 108: 689-701). We therefore tested the effect of S1P1 receptor antagonists on the barrier enhancement response of endothelial cell monolayers to HDL. Both the S1P1 antagonist, 857390, and the S1P1/S1P3 antagonist, VPC23019, inhibited the TEER response to HDL as well as the TEER response to S1P (see FIGS. 3C and D).

HDL stimulates Erk and Akt activation in endothelial cells. S1P signaling in endothelial cells involves Erk1/2 and Akt activation (Taha, T. A., et al., Biochim Biophys Acta 1682: 48-55). We performed multiplex microbead suspension array analysis to evaluate the effect of HDL on the activation of Erk1/2 and Akt, signaling pathway intermediates that have been implicated in endothelial barrier function (Vogel, C., et al., Journal of cellular physiology 212: 236-243; Vogel, C., Journal of cellular physiology 212: 236-243; Lee, J. F., et al., J Biol Chem 281: 29190-29200). As shown in FIG. 4A, S1P elicits a transient increase in Erk1/2 phosphorylation with peak levels detectable within 3-5 min of treatment. The response to S1P was dose dependent (see FIG. 4B). Similarly, HDL produced a transient increase in Erk1/2 phosphorylation with peak phosphorylation detectable within 3-5 min of treatment (see FIG. 4C). The activation response to HDL was also dose dependent, reaching a maximal plateau at 333 μg/ml (based on LC-MS-MS analysis, this amount of HDL contained 135 nM S1P) (see FIG. 4D). Similar effects of HDL and S1P on the activation of Akt were also observed (see FIG. 4E-H). Furthermore, the findings indicate that when equimolar amounts of S1P carried either on HDL or albumin they elicit a similar magnitude of Erk and Akt activation (compare FIG. 4B with FIG. 4D and FIG. 4F with FIG. 4H).

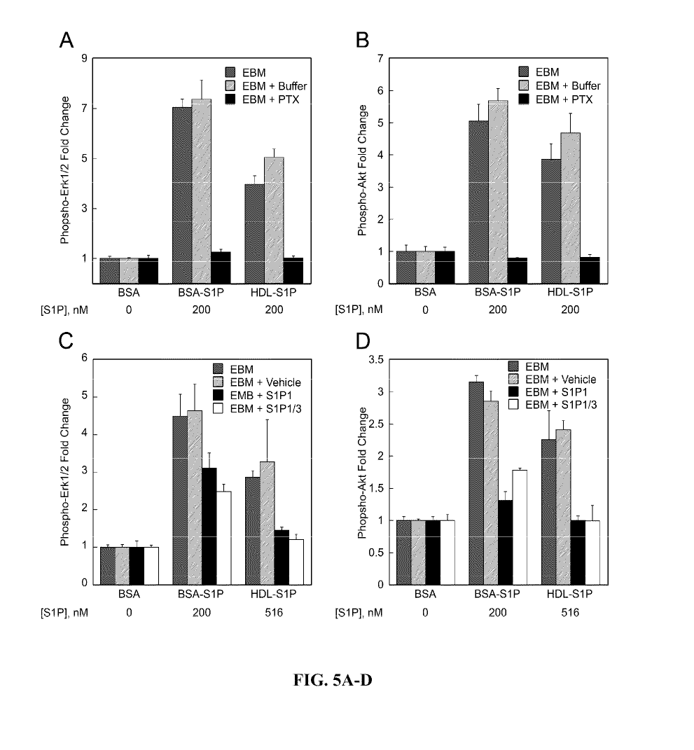

Activation of Erk1/2 and Akt by HDL is blocked by pertussis toxin and a S1P1 antagonist. Previous studies have shown that S1P-mediated activation of Erk and Akt is PTX sensitive (Park, K. S., et al., Biochem Biophys Res Commun 356: 239-244; Wu, J., et al., J Biol Chem 270: 11484-11488) and mediated by the S1P receptor, S1P1 (Lee, M. J., et al., Mol Cell 8: 693-704; Osinde, M., et al., Neuropharmacology 52: 1210-1218). Multiplex microbead suspension array analysis was performed to evaluate the effect of PTX on HDL-induced activation of Erk1/2 and Akt in endothelial cells. The activation of Akt and Erk1/2 by either HDL or S1P was inhibited by PTX (see FIGS. 5A and B), suggesting the requirement for Gᵢ-coupled S1P receptors. The S1P receptor, S1P1, was implicated in this process by the finding that the S1P1 antagonist, 857390 and the S1P1/S1P3 antagonist, VPC23019, inhibited HDL-induced and S1P-induced activation of Erk1/2 and Akt (see FIGS. 5C and D).

S1P levels in blood inversely correlate with cardiovascular disease. Epidemiological data from the Framingham Heart Study (Gordon, T., et al., Archives of internal medicine 141: 1128-1131) and other prospective studies demonstrate an inverse correlation between plasma levels of HDL and risk of cardiovascular disease. Interestingly, there are some individuals with high HDL levels and normal LDL levels that have cardiovascular disease. Based on the emerging concept that S1P is the mediator of many of the cardioprotective effects of HDL, we hypothesized that the failure of high plasma levels of HDL to be cardioprotective in some individuals might be due to a deficiency in HDL-associated S1P. If this hypothesis is true then S1P levels in HDL should inversely correlate with occurrence of cardiovascular disease. To test this hypothesis, we sought to use liquid chromatography/mass spectrometry (LC-MS-MS) to measure S1P levels in plasma lipoprotein samples from a group of individuals with high HDL cholesterol (>80 mg/dl) and verified ischemic heart disease (IHD) and in control samples from gender and age matched individuals with high HDL-C levels, but no evidence of cardiovascular disease/coronary heart disease.

HDL compositional analyses (i.e., levels of triglycerides, phospholipids, apoA-I and apoE in HDL subfractions) has been performed on samples from the Copenhagen City Heart Study (CCHS, a prospective study of a cohort of persons randomly selected from the population of the city of Copenhagen) (31, 32) of individuals having high and low HDL, with and without evidence of cardiovascular disease (ischemic heart disease). The characteristics of subjects from which samples were derived are summarized in Table 1. As described herein LC-MS-MS sphingolipid analysis was performed on the samples specifically to look for differences in S1P levels.

204 CCHS serum samples that had been freed of apolipoprotein B (apoB)-containing particles by dextran-sulfate/MgCl₂ precipitation (Polymedco) and thus can be considered LDL poor, HDL-containing preparations were prepared. Blinded LC-MS-MS sphingolipid analysis on the samples. Following completion of the LC-MS-MS analysis, we learned that the four groups of CCHS samples that we tested included 55 samples from individuals with high HDL-C (females:≧73.5 mg/dL; males:≧61.9 mg/dL) and no evidence of IHD, 53 samples from individuals with high HDL-C and evidence of IHD, 54 samples from individuals with low HDL-C (females:≦38.7 mg/dL; males:≦34.1 mg/dL) and no evidence of IHD and 42 samples from individuals with low HDL-C and evidence of IHD (see Table 1). All individuals had LDL-C<160 mg/dL, triglycerides<150 mg/dL, and none were treated with LDL-C-lowering medications. Subjects had an average age of 62 years, approximately 30% were women and 8.5% had diabetes mellitus.

Pairwise comparisons were performed between the groups of LC-MS-MS data and Tukey's adjustment for multiple comparisons was used to control the Type I error rate associated with the pairwise comparisons. The one-way ANOVA model was fit using proc glm in SAS v9.1.3. The studentized residuals were calculated and assessed for the model assumptions of normality and constant variance. It was further assumed that the samples are independent of one another. Hypothesis tests were conducted at level of significance 0.05.

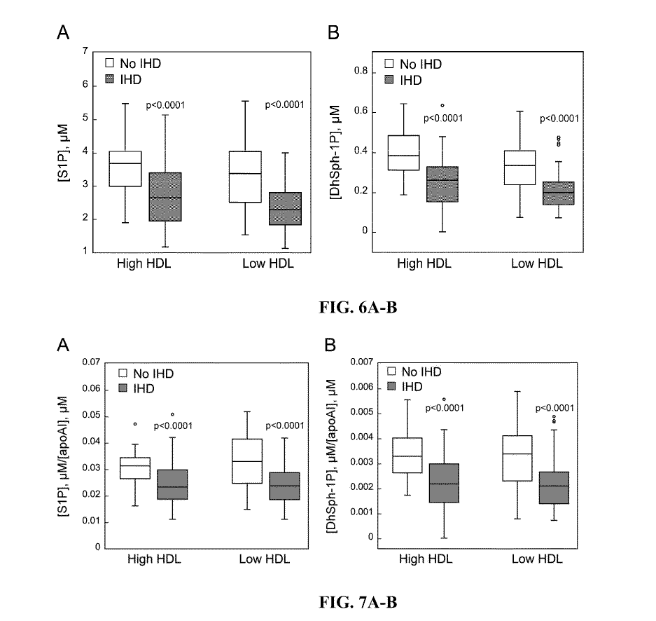

Based on the results showed that S1P levels were significantly lower (p<0.0001) in samples from individuals with high HDL-C having IHD as compared to individuals with high HDL-C having no evidence of cardiovascular disease (see FIG. 6A). Furthermore, S1P levels were significantly lower (p<0.0001) in samples from individuals with low HDL-C with IHD as compared to individuals with low HDL-C having no evidence of cardiovascular disease (see FIG. 6A). When S1P levels were adjusted relative to the concentration of apolipoprotein A-I (apoA-I) in the samples there was also a significantly lower ratio (p<0.0001) of [S1P] μM/[apoAI] μM in samples from individuals with high HDL-C having IHD as compared to individuals with high HDL having no evidence of IHD (see FIG. 7A). Other sphingolipids were also evaluated in our studies and similar to S1P, dihydro-S1P (DH-S1P) (see FIG. 6B) and C24:1-ceramide levels were found to inversely correlate with IHD (not shown). By contrast, levels of several other sphingolipids including, C22 ceramide, C22:1 ceramide and C24 ceramide were not different between subjects with and without IHD in either the high or low HDL-C groups (not shown).

Summary: The results show that HDL-sphingolipid levels are important in deciphering the protective role of HDL in IHD.

### 4. Example 4

### HDL and Reconstituted-HDL Enhance Transendothelial Electrical Resistance in a Manner Related to Associated S1P Levels

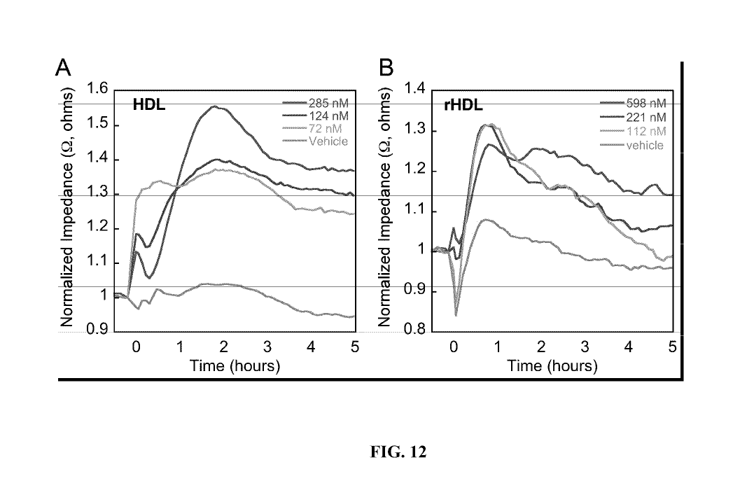

As shown in FIG. 12 the HDL and reconstituted-HDL enhance transendothelial electrical resistance in a manner related to associated S1P levels. S1P-augmented HDL was prepared by preincubation of native HDL with S1P followed by dialysis against 0.03 mM EDTA in Dulbecco's PBS to remove free S1P. For reconstituted-HDL (rHDL) preparations, lipids were extracted from native HDL using diethyl ether and reconstituted using a 1:100 molar ratio of delipidated HDL:POPC according to the cholate dialysis method of Matz and Jonas. JBC 257(8) 1982. A minimal TEER plateau was reached within ˜24 h of replacing culture medium of confluent ECs with serum free medium. Monolayers were incubated with 250 μg/ml S1P-augmented HDL (A) or rHDL (B) containing varying amounts of S1P as indicated in figure legend. Impedance values were normalized by dividing each value by the level of impedance measured just prior to the addition of effectors.

### 5. Example 5

### Determining HDL-S1P Levels in Assessing the Relative Risk for Cardiovascular Disease

Rationale: Described herein it is shown that S1P levels (as well as DH-S1P and C24:1-ceramide levels) in HDL preparations from individuals with HDL-C levels above and below the normal range correlate inversely with the occurrence of IHD. An important direction for this research is to determine the prognostic value of HDL-S1P levels in assessing the relative risk for cardiovascular disease. Chronically low levels of HDL-associated S1P can contribute to both the etiology and progression of atherosclerosis. A substantial body of evidence indicates that S1P signaling mediates a host of cardioprotective effects (Argraves, K. M., J Lipid Res 48: 2325-2333). These effects are expected to be diminished in individuals with chronically low lipoprotein-associated S1P (e.g., HDL-S1P). Identification of HDL-associated S1P as a new biomarker is particularly important in assessing risk in those individuals with susceptibility to IHD, yet lack conventional risk factors such as elevated LDL-C or low HDL-C.

Approach: Described herein is as a type of retrospective cohort design, where a single baseline sample is used to define the risk factor groups. The first examination of CCHS subjects will serve as the baseline target population; a random sample of 130 subjects will be selected from those subjects who were free of IHD at baseline. Each sample will be submitted for sphingolipid analysis, and the S1P level recorded. In addition, status with respect to IHD diagnosis following the first examination will be determined.

It is shown herein that low levels of HDL-associated S1P is associated with an increased risk of developing IHD. A χ² test with level of significance 0.05 was used for the purposes of this analysis. S1P levels are dichotomized using the sample median as the cutpoint. The χ² test involves a sample size requirement based on the expected cell counts; if this requirement is not met, Fisher's exact test will be conducted. In either case, a significant result indicates that there is an association between S1P and the incidence of IHD. The magnitude of the association will be estimated using the relative risk, and the corresponding 95% confidence interval will be constructed.

Based on the analysis method described above, it is estimated that a total sample size of n=130 would be needed to detect a difference in proportions of 0.25 with at least 80% power, at a two-sided level of significance of 0.05. Assuming that the proportion of subjects developing disease in the unexposed (high S1P) group is 0.5, which is the worst-case scenario for the variability of a binomial proportion, this effect size would correspond to a proportion of subjects developing disease in the exposed group of 0.75 and a relative risk of 1.5.

HDL fractions can be evaluated that have been prepared by subjecting serum to magnetic bead-dextran-sulfate/MgCl₂ precipitation (Reference Diagnostics, Inc., Bedford, Mass.) to remove apoB lipoproteins (i.e., LDL and VLDL). The total cholesterol levels in the HDL preparations was measured from magnetic bead-dextran-sulfate/MgCl₂ precipitation of CCHS serum samples. On average, total cholesterol levels in these HDL preparations were found to be within 10% of the HDL-C levels measured in total serum. Thus, there is not a large loss of HDL as a result of the magnetic bead-dextran-sulfate/MgCl₂ precipitation.

The HDL samples were analyzed by the Analytical Lipidomics Core at MUSC for sphingolipid analysis. This core provides qualitative (compound identification) and quantitative LC-MS-MS analysis of the key sphingolipids from different biological materials (cells, tissues, serum, blood) for determining their basal levels and changes in response to the exogenous agents. Analyses include: sphingoid bases (i.e., sphingosine, sphinganine, phytosphingosine), spingoid base phosphates (i.e., S1P, dihydro-S1P), ceramide, dihydroceramide, OH ceramide, phytoceramide species, ceramide phosphate species, and sphingomyelin species. The url for Analytical Lipidomics Core facility website is: http://hcc.musc.edu/research/sharedresources/lipidomics/lipidomicsanalytics.htm. The analytical equipment of the core includes a ThermoFinnigan TSQ 7000™ triple-stage quadrupole mass spectrometer, a ThermoFinnigan LCQDUO ion trap mass spectrometer, a SCIEX Q-Trap, triple quadrupole/ion trap combination instrument. These instruments use dual ionization mode: electrospray ionization and atmospheric pressure chemical ionization and offer outstanding sensitivity with low detection limits. LC-MS-MS can be used to quantify sphingolipid levels in total serum and lipoprotein samples (i.e., HDL and LDL) ranging in concentration from 1-35 mg/ml using as little as 10 μl.

Impact: Currently, HDL cholesterol is the only component of HDL clinically assessed to determine a subject's risk for cardiovascular disease. However, as described herein HDL associated sphingolipids is a risk factor then assessment of HDL associated sphingolipids can improve the ability to target those in need of intervention.

### 6. Example 6

### Determine S1P Levels in HDL and/or Albumin that Inversely Correlate with Occurrence of IHD

Rationale: Although HDL is a major carrier of S1P in blood it is not the exclusive carrier. Indeed, S1P is found in association with LDL, VLDL and albumin (Murata, N., et al., Biochem J 352 Pt 3: 809-815). As described herein S1P levels in serum that has been freed of apoB-containing lipoproteins (i.e., LDL and VLDL) by magnetic bead-dextran-sulfate/MgCl₂ precipitation, inversely correlate with IHD. These findings indicates that S1P associated with HDL can be atheroprotective. However, serum albumin is also an S1P-binding apolipoprotein and the dextran-sulfate/MgCl₂ precipitation procedure that we used to remove LDL and VLDL does not remove albumin. Importantly, epidemiological studies have shown that a highly significant inverse relationship exists between serum albumin levels and risk of coronary heart disease (Kuller, L. H., et al., American journal of epidemiology 134: 1266-1277). Furthermore, all of the putative cardioprotective effects reported for S1P have been experimentally demonstrated using albumin as the S1P carrier (Argraves, K. M., et al., J Lipid Res 48: 2325-2333). Thus, there is a dual need to: 1) establish that S1P levels in HDL preparations free of albumin correlate inversely with IHD; and 2) establish whether levels of albumin-S1P in blood correlate inversely with IHD. As described herein both HDL-S1P and albumin-S1P levels in blood correlates inversely with occurrence of IHD. It is important to note that both HDL-S1P and albumin-S1P exhibit similar effects on endothelial barrier enhancement and S1P receptor-dependent activation of Erk1/2 and Akt (see FIG. 3-5).

Determine S1P Levels in HDL-Containing/Albumin-Free Fractions of Serum

Approach: LC-MS-MS analysis can be performed on CCHS serum samples that have been processed to remove both apoB-containing lipoproteins (i.e., LDL and VLDL) and albumin Briefly, serum samples will be subjected to magnetic bead-dextran-sulfate/MgCl₂ precipitation to remove apoB-containing lipoproteins and subsequently freed of albumin by absorption against immobilized Cibacron Blue F3GA (SwellGelBlue Albumin Removal Kit from Pierce Biotechnology, Inc) (Nakajima, K., et al., Clinica chimica acta; international journal of clinical chemistry 223: 53-71) Immobilized Cibacron Blue F3GA is a routinely used dye-ligand affinity matrix for purification of albumin, enzymes (including NAD+ and NADP+), coagulation factors, interferons and related proteins. Cibacron Blue has no reported binding affinity for apolipoproteins including apoA-I or apoB. The effectiveness of albumin removal will be evaluated on the processed serum fractions using the Agilent 2100 bioanalyzer with the Protein 200 Plus LabChip Kit (Pierce Biotechnology). The SwellGelBlue system can be used with 10-100 μl of human serum and the Agilent analyzer only requires 4 μl sample volume. The LC-MS-MS measurements of S1P are routinely performed on 50 μl of serum or plasma.

The HDL-containing/albumin-free fractions to be tested will be derived from the sera of the same CCHS subject group (see Table 3). Subjects with IHD demonstrates lower HDL-associated S1P levels than subjects without. The data will be analyzed statistically using independent samples t-test with level of significance 0.05. The validity of the equal variances assumption can be assessed, and the unequal variances t-test can be conducted if appropriate.

TABLE 3

| | With IHD | | Without IHD | |
|---|---|---|---|---|
| | Group A | Group B | Group C | Group D |
| | (high HDL) | (low HDL) | (high HDL) | (low HDL) |
| | (n = 58) | (n = 47) | (n = 58) | (n = 55) |
| Age, years | 62.9 ± 10.3 | 60.7 ± 9.2 | 62.9 ± 10.2 | 62.4 ± 9.7 |
| Total | 208.3 ± 26.7 | 181.9 ± 30.6 | 206.7 ± 31.6 | 166.9 ± 30.9 |
| Cholesterol, | | | | |
| mg/dl | | | | |
| HDL, mg/dl | 78.8 ± 14.2 | 31.8 ± 5.3 | 80.3 ± 14.3 | 33.7 ± 5.8 |
| LDL, mg/dl | 112.4 ± 26.2 | 120.9 ± 30.1 | 118.0 ± 28.1 | 117.9 ± 25.4 |
| Triglycerides. | 82.7 ± 29.5 | 105.0 ± 30.8 | 74.8 ± 24.8 | 107.7 ± 29.1 |
| mg/dl | | | | |
| Body mass | 24.9 ± 4.1 | 29.2 ± 3.6 | 23.5 ± 3.2 | 27.9 ± 5.1 |
| index, kg/m² | | | | |
| Smokers, % | 30.2 | 30.4 | 45.6 | 34.5 |
| Diabetes | 8.8 | 12.8 | 5.2 | 7.3 |
| mellitus, % | | | | |

All Values are original measurements from the above mentioned studies. Selection and matching for the present study were based on these values. All individuals had LDL-C <160 mg/dL, tryglycerides <150 mg/dL, and none were treated with LDL-C-lowering medications. Group A: Females n = 17, Males n = 41; Group B: Females n = 13, Males n = 34; Group C: Females n = 17, Males n = 41; Group D: Females n = 16, Males n = 39. Group A had high HDL-C (>90 percentile) and verified IHD; this group was compared with Group C without IHD, but matched by age, sex, and similar HDL-C levels. Group B had low HDL-C but matched by age, sex, and similar HDl-C levels. All individuals without IHD were selected from the Copenhagen City Heart Study's 4^{th} examination. Patients with IHD were selected from individuals referred to the Copenhagen University Hospital, Rigshospitalet, Denmark from coronary angiography.

Expected results: The dextran-sulfate/MgCl₂ precipitation and Cibacron Blue chromatography procedures are expected to effectively remove LDL, VLDL and albumin with minimal loss in serum sample volume. As a result, the samples will contain HDL and little or no other known S1P lipoprotein carriers. Since there is no reason to believe that the albumin S1P fraction of serum exclusively accounts for the observed inverse correlation with IHD, the S1P levels in the ‘HDL-containing, albumin-free, LDL/VLDL-free serum fractions’ are expected to correlate inversely with occurrence of IHD.

Based on published information (Murata, N., K. et al., Biochem J 352 Pt 3: 809-815). depletion of LDL, VLDL and albumin from serum can be expected to leave ˜54% of total blood S1P in the resulting HDL-containing fraction. Current LC-MS-MS analysis methods in reducing the level of S1P in test samples by 50% will permit S1P quantification well above baseline detection levels.

Determine S1P Levels in the Albumin-Containing Fraction of CCHS Serum.

Approach: LC-MS-MS sphingolipid analysis can be performed on serum samples that have been freed of HDL, LDL and VLDL but retain the only other known serum carrier of S1P, albumin. The serum samples to be employed in these experiments will be from the CCHS subjects whose serum was previously evaluated by LC-MS-MS (see FIGS. 6 and 7). Group A samples from individuals with high HDL-C [>80 mg/dl] and IHD and Group C samples from gender and age matched individuals with high HDL but no evidence of IHD). Briefly, serum samples can be subjected to dextran-sulfate/MgCl₂ precipitation to remove apoB-containing lipoproteins and subsequently freed of HDL by absorption against Sepharose immobilized anti-apoA-I IgG as described by others (Nakajima, K., et al., Clinica chimica acta; international journal of clinical chemistry 223: 53-71). The effectiveness of the immunoabsorption process can be evaluated by anti-apoA-I and anti-apoB ELISA using aliquots of serum samples before and after absorption. The corresponding hypothesis can be tested as described elsewhere herein, using an independent samples t-test with level of significance 0.05.

Outlook and alternative approaches: The magnetic bead-dextran-sulfate/MgCl₂ precipitation and anti-apoA-I IgG affinity chromatography procedures are expected to effectively remove LDL, VLDL and HDL. As a result, the samples will contain albumin and little or no other known S1P lipoprotein carriers. The procedures allow for rapid removal of lipoproteins from the CCHS samples with minimal loss of volume. Based on the fact that ˜35% of the S1P in blood is associated with the non-lipoprotein fraction and that albumin is the major component of this fraction, then it is reasonable to conclude as much 35% of blood S1P is associated with albumin. If S1P levels in albumin and HDL are generally modulated coordinately then it can be expected that individuals with high HDL-S1P levels will have high albumin-S1P levels. Conversely, individuals with low HDL-S1P levels will have low albumin-S1P levels. In this case, an inverse correlation could be found to exist between albumin-S1P levels and IHD. This result can be consistent with epidemiological studies that show a highly significant inverse relationship to exist between serum albumin levels and risk of coronary heart disease (Kuller, L. H., American journal of epidemiology 134: 1266-1277). If S1P levels on HDL and albumin are not modulated coordinately then an inverse correlation with IHD could exists only for HDL-S1P levels or albumin-S1P levels. Given the similarities in the signaling activities of HDL-S1P and albumin-S1P (Argraves, K. M., et al., J Biol Chem 283: 25074-25081) and given that an inverse relationship exists between serum albumin levels and risk of coronary heart disease it is expected that S1P levels in the albumin fraction will correlate inversely with occurrence of IHD.

As an alternative to the analysis of serum after sequential removal of LDL, VLDL and HDL, the albumin-bound sphinoglipids associated can be analyzed as described elsewhere herein with the immobilized Cibacron Blue F3GA. The impact of Cibacron Blue F3GA on LC-MS-MS based quantification of sphingolipids is under current investigation. This approach would significantly streamline the analyses and reduce the amount of serum that would be required from the CCHS.

Determine the Impact of Low Levels of Lipoprotein-Associated S1P on the Process of Atherosclerosis in a Mouse Model.

Approach: As an approach to assess the impact of low levels of lipoprotein-associated S1P on the process of atherosclerosis, a transgenic mouse model of plasma S1P deficiency was used. Mice carrying a compound mutation of genes encoding the two S1P-producing enzymes, sphingosine kinase 1 (Sphk1) and sphingosine kinase 2 (Sphk2) (i.e., Sphk1^{-/-}/Sphk2^{+/-} mice) display ˜3-fold reduced levels of plasma S1P (Venkataraman, K., et al., Circ Res 102: 669-676). The susceptibility can be assessed of the Sphk1^{-/-}/Sphk2^{+/-} mice (age and sex matched) to high fat diet-induced atherosclerosis. Atherosclerosis in the aortas of the mice will be measured by quantification of oil red O-positive lesions (Paigen, B., et al., Atherosclerosis 68: 231-240). This analysis can be performed for lesions both within the aortic sinus and throughout the aorta. The mice can also be breed onto the apoE knockout background and evaluate the compound mutants (i.e., Sphk1^{-/-}/Sphk2^{+/-}/ApoE^{-/-} mice) for susceptibility to atherosclerosis as compared to controls.

Outcome: A low level of HDL-associated S1P is a risk factor for IHD/atherosclerosis, thus, S1P-deficient mice (e.g., Sphk1^{-/-}/Sphk2^{+/-}/ApoE^{-/-} mice) can develop atherosclerosis at an accelerated rate as compared to wild-type controls.

New direction: The impact of infused synthetic HDLs containing S1P on atherosclerosis in mouse models can also be performed. Material and methods for this direction are described elsewhere herein.

### 7. Example 7

### Determining HDLs from Subjects with Low HDL-Associated S1P that are Dysfunctional with Respect to S1P Signaling

Rationale: Alteration in endothelial barrier function is a factor underlying post ischemic edema, the recruitment and migration of monocytes as well as the introduction of triglyceride rich lipoprotein particles into the intima of the blood vessel (Nordestgaard, B. G., et al., Arterioscler Thromb Vasc Biol 15: 534-542). As described herein (Argraves, K. M., et al., J Biol Chem 283: 25074-25081), it is established that HDL can regulate endothelial barrier integrity as evidenced by its ability to increase TEER in the ECIS assay (see FIG. 8). The TEER response to HDL is dose dependent and S1P receptor-dependent, but it remains to be established that the TEER/endothelial barrier response is directly correlated to concentration of S1P associated with HDL. Establishing this relationship would be evidence in support of the hypothesis that the atheroprotective activity of HDL is function of its S1P levels, with higher levels being protective.

Measure Endothelial Barrier Promoting Activity of HDLs from Subjects with Low HDL-Associated S1P as Well as the Barrier Promoting Activity of Synthetic HDLs Containing Varying Amounts of S1P.

Approach: The HDL samples to be employed in are be selected from the CCHS subjects whose serum discussed and evaluated elsewhere herein by LC-MS-MS (i.e., Group A samples from individuals with high HDL-C (>80 mg/dl) and IHD and in Group C samples from gender and age matched individuals with high HDL-C but no evidence of IHD). From these groups serum samples can be obtained from subjects whose HDL-associated S1P levels span a range from high to low based on analysis discussed elsewhere herein using LC-MS-MS analysis data.

To prepare HDL from CCHS serum samples, the sera will first be subjected to magnetic bead-dextran-sulfate/MgCl₂ precipitation (Reference Diagnostics, Bedford, Mass.) to remove apoB-containing particles (i.e., LDL and VLDL) (Warnick, G. R., et al., Clin Chem 28: 1379-1388). The resulting HDL-containing preparations will be subjected to absorption on immobilized Cibacron Blue to remove albumin. The effectiveness of these procedures will be assessed using the apoB and albumin ELISAs and Agilent 2100 bioanalyzer using the Protein 200 Plus LabChip Kit. The resulting albumin- and apoB lipoprotein-depleted HDL-containing fractions will be evaluated for their ability to influence transendothelial electrical resistance (TEER) using the Electrical Cell Substrate Impedance Sensing (ECIS) Assay (Finigan, J. H., et al., J Biol Chem 280: 17286-17293; Garcia, J. G., et al., J Clin Invest 108: 689-701). TEER, an index of endothelial cell barrier function, will be measured using an ECIS Model 1600 instrument (Applied Biophysics, Troy, N.Y.).

Synthetic HDLs: Synthetic HDLs to be evaluated by ECIS assay with varying concentrations of associated S1P at high and low physiological levels. Briefly, synthetic discoidal HDL containing palmitoyl-oleoyl-phosphatidylcholine (POPC; (Avanti Polar Lipids, Alabaster, Ala.), apoA-I plus and minus S1P (Avanti Polar Lipids) will be prepared by the cholate dialysis method (Matsuo, Y., Atherosclerosis.) The amount of S1P incorporated into the synthetic HDLs will be adjusted to cover the range of S1P levels found in the HDL fractions of the CCHS subject sera, as measured by LC-MS-MS.

ECIS: Electrical cell substrate impedance sensing (ECIS) assays will be conducted essentially as described in (1). Briefly, 8W10E+ electrode arrays will be pre-coated with human fibronectin (Invitrogen, Carlsbad, Calif.) at 100 μg/ml in 0.15M NaCl, 0.01M Tris, pH 8.0. HUVECs in EGM-2 medium will be seeded into the wells at a density of 1×10⁵ cells per well. Electrical resistance (impedance) will be measured every 5 mM at a frequency of 15 kHz. For every 48 h of culture, 50% volume of medium will be replaced with fresh EGM-2. When the electrical resistance reaches a maximal plateau (−3 days) the medium will be replaced with serum-free endothelial basal medium (EBM; Lonza) containing 1× penicillin-streptomycin-glutamine (Invitrogen). Electrical resistance will be monitored until a minimal plateau is reached (˜24 h). Either HDL from CCHS subjects (depleted of albumin and apoB lipoproteins) or synthetic HDLs will be introduced into the culture medium by removing a volume corresponding to that of the HDL to be added. The maximum volume of each HDL added will not exceed 1/25 of the 400 μl volume of conditioned culture medium in each well. For experiments evaluating the effects of pertussis toxin (PTX), PTX (100 ng/ml) or the PTX buffer (50% glycerol with 50 mM Tris, 10 mM glycine, and 0.5M NaCl, pH 7.5) will be added to EBM during the final 12 h of serum starvation. S1P or HDL will then be added to the medium. Electrical resistance will be monitored through 5 h post addition of agents.

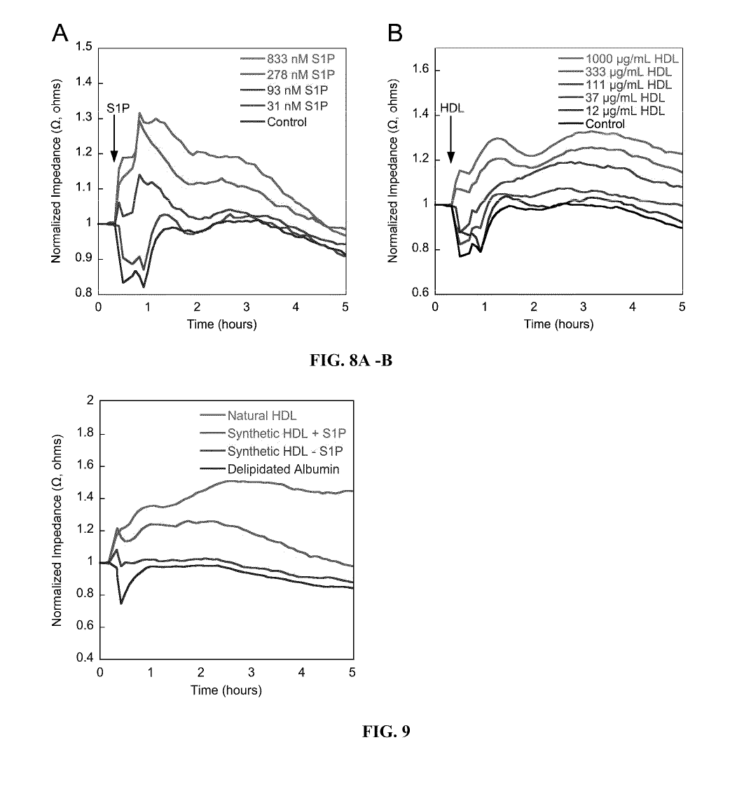

Outcome and alternative approaches: A correlation between the capacity of HDLs to promote TEER and their concentration of S1P is expected such that HDLs with low levels of S1P will have a reduced capacity to stimulate TEER. As described herein it is demonstrated that synthetic HDLs containing S1P augment TEER whereas synthetic HDLs made without exogenously added S1P do not (see FIG. 9). There is a possibility that bather responses can differ depending on the type of endothelial cell tested, as there is ample evidence to indicate molecular diversity among endothelial cells from different tissues, particularly the heart. Therefore, considering that coronary arteries are affected in IHD, the effects of synthetic HDLs on TEER in human coronary artery endothelial cells should be measured (Lonza) in addition to HUVECs. Additionally, since albumin-S1P levels inversely correlate with IHD, studies will be performed to test the effects of the albumin-S1P fraction of CCHS serum samples on TEER.

Define the Potential of HDLs from Subjects with Low HDL-Associated S1P and the Potential of Synthetic HDLs Containing Varying Amounts of S1P to Activate of Map Kinase and Akt Signaling Pathways in Endothelial Cells.

Approach: A bead-based multiplex assay will be used to determine changes in phosphorylated Erk1/2, phosphorylated Akt, and total Erk and total Akt in endothelial cells in response to treatments with HDLs from dislipidemic subjects and synthetic HDLs containing defined amounts of S1P. These assays will be conducted essentially as described by (1). Briefly, HUVECs will be seeded into 24 well dishes (Corning) at 2×10⁵ cells per well and grown for 18 h in endothelial growth medium-2 (EGM-2, Lonza). The medium will then be replaced with endothelial basal medium (EBM) containing 0.1% FBS and the cells grown for 24 h. The medium will then be supplemented with HDL samples (i.e., CCHS subject serum-derived HDL freed of albumin- and apoB-containing lipoproteins) or synthetic HDLs (containing defined amounts of S1P). As described above, this will be done by removing a volume of the conditioned culture medium corresponding to that of the HDL to be added. The cells will be extracted with Bio-Plex cell lysis buffer (Bio-Rad, Hercules, Calif.) and protein concentration determined by Bio-Rad DC Protein Assay (Bio-Rad). Levels of phospho-Erk1/2, total-Erk1/2 and phospho-Akt in the extracts will be determined by multiplex bead assay using kits (Bio-Rad) and a Bioplex-200 instrument (Bio-Rad).

Outcome and alternative approaches: A correlation between the capacity of HDLs to promote endothelial cell Erk1/2 and Akt activation is expected and their concentration of S1P such that HDLs with low levels of S1P will have a reduced capacity to stimulate Erk1/2 and Akt activation. These experiments will involve testing aliquots from the Group A and C-derived HDL-containing fractions freed of albumin and apoB lipoproteins as described above (i.e., Group A samples from individuals with high HDL-C and IHD and in Group C samples from individuals with high HDL-C but no evidence of IHD). As described herein it is established that a change in pErk1/2 relative to controls with a concentration of HDL can be detected as low 111 μg protein/ml (see FIG. 4D). The mean concentration of HDL protein in the CCHS subjects is 1910 μg/ml. Therefore, serum samples should be diluted ˜17 fold to treat HUVECs. Since the volume of medium required to culture HUVECs in a 1.9 cm² well is 400 μA then 23 μl of CCHS subject serum should be added (freed of albumin and apoB lipoproteins). Additionally, since albumin-S1P is a component of serum that inversely correlates with IHD then the signaling effects of the albumin fraction should be evaluated from CCHS samples (i.e., serum freed of apoB and apoA-I lipoproteins by dextran sulfate/MgCl₂ precipitation followed by anti-apoA-I affinity chromatography).

### REFERENCES

1. Pettus B J, Bielawska A, Kroesen B J, Moeller P D, Szulc Z M, Hannun Y A, Busman M. Observation of different ceramide species from crude cellular extracts by normal-phase high-performance liquid chromatography coupled to atmospheric pressure chemical ionization mass spectrometry. Rapid Commun Mass Spectrom. 2003; 17(11):1203-1211.
2. Pettus B J, Kroesen B J, Szulc Z M, Bielawska A, Bielawski J, Hannun Y A, Busman M. Quantitative measurement of different ceramide species from crude cellular extracts by normal-phase high-performance liquid chromatography coupled to atmospheric pressure ionization mass spectrometry. Rapid Commun Mass Spectrom. 2004; 18(5):577-583.
3. Okajima F. [Establishment of the method for the measurement of sphingosine-1-phosphate in biological samples and its application for S1P research]. Nippon Yakurigaku Zasshi. 2001; 118(6):383-388.
4. Murata N, Sato K, Kon J, Tomura H, Okajima F. Quantitative measurement of sphingosine 1-phosphate by radioreceptor-binding assay. Anal Biochem. 2000; 282(1):115-120.
5. Lee Y M, Venkataraman K, Hwang S I, Han D K, Hla T. A novel method to quantify sphingosine 1-phosphate by immobilized metal affinity chromatography (IMAC). Prostaglandins & other lipid mediators. 2007; 84(3-4):154-162.
6. Yatomi, Y., Y. Igarashi, L. Yang, N. Hisano, R. Qi, N. Asazuma, K. Satoh, Y. Ozaki, and S. Kume. 1997. Sphingosine 1-phosphate, a bioactive sphingolipid abundantly stored in platelets, is a normal constituent of human plasma and serum. Journal of biochemistry 121: 969-973.
7. Murata, N., K. Sato, J. Kon, H. Tomura, M. Yanagita, A. Kuwabara, M. Ui, and F. Okajima. 2000. Interaction of sphingosine 1-phosphate with plasma components, including lipoproteins, regulates the lipid receptor-mediated actions. Biochem J 352 Pt 3: 809-815.
8. Argraves, K. M., and W. S. Argraves. 2007. HDL serves as an S1P signaling platform mediating a multitude of cardiovascular effects. J Lipid Res 48: 2325-2333.
9. Argraves, K. M., P. J. Gazzolo, E. M. Groh, B. A. Wilkerson, B. S. Matsuura, W. O. Twal, S. M. Hammad, and W. S. Argraves. 2008. High density lipoprotein-associated sphingosine 1-phosphate promotes endothelial bather function. J Biol Chem 283: 25074-25081.
10. Gordon, T., W. P. Castelli, M. C. Hjortland, W. B. Kannel, and T. R. Dawber. 1977. High density lipoprotein as a protective factor against coronary heart disease. The Framingham Study. The American journal of medicine 62: 707-714.
11. Gordon, T., W. B. Kannel, W. P. Castelli, and T. R. Dawber. 1981. Lipoproteins, cardiovascular disease, and death. The Framingham study. Archives of internal medicine 141: 1128-1131.
12. Gordon, D. J., J. L. Probstfield, R. J. Garrison, J. D. Neaton, W. P. Castelli, J. D. Knoke, D. R. Jacobs, Jr., S. Bangdiwala, and H. A. Tyroler. 1989. High-density lipoprotein cholesterol and cardiovascular disease. Four prospective American studies. Circulation 79: 8-15.
13. Ansell, B. J., M. Navab, S. Hama, N. Kamranpour, G. Fonarow, G. Hough, S. Rahmani, R. Mottahedeh, R. Dave, S. T. Reddy, and A. M. Fogelman. 2003. Inflammatory/antiinflammatory properties of high-density lipoprotein distinguish patients from control subjects better than high-density lipoprotein cholesterol levels and are favorably affected by simvastatin treatment. Circulation 108: 2751-2756.
14. Schnohr, P., G. Jensen, J. Nyboe, and A. Eybjaerg Hansen. 1977. [The Copenhagen City Heart Study. A prospective cardiovascular population study of 20,000 men and women]. Ugeskrift for laeger 139: 1921-1923.
15. Schnohr, P., J. S. Jensen, H. Scharling, and B. G. Nordestgaard. 2002. Coronary heart disease risk factors ranked by importance for the individual and community. A 21 year follow-up of 12 000 men and women from The Copenhagen City Heart Study. European heart journal 23: 620-626.
16. Juul, K., A. Tybjaerg-Hansen, R. Steffensen, S. Kofoed, G. Jensen, and B. G. Nordestgaard. 2002. Factor V Leiden: The Copenhagen City Heart Study and 2 meta-analyses. Blood 100: 3-10.
17. Warnick, G. R., J. Benderson, and J. J. Albers. 1982. Dextran sulfate-Mg2+ precipitation procedure for quantitation of high-density-lipoprotein cholesterol. Clin Chem 28: 1379-1388.
18. Bielawski, J., Z. M. Szulc, Y. A. Hannun, and A. Bielawska. 2006. Simultaneous quantitative analysis of bioactive sphingolipids by high-performance liquid chromatography-tandem mass spectrometry. Methods 39: 82-91.
19. Bu, S., M. Yamanaka, H. Pei, A. Bielawska, J. Bielawski, Y. A. Hannun, L. Obeid, and M. Trojanowska. 2006. Dihydrosphingosine 1-phosphate stimulates MMP1 gene expression via activation of ERK1/2-Ets1 pathway in human fibroblasts. Faseb J 20: 184-186.
20. Lemaitre, V., T. K. O'Byrne, A. C. Borczuk, Y. Okada, A. R. Tall, and J. D'Armiento. 2001. ApoE knockout mice expressing human matrix metalloproteinase-1 in macrophages have less advanced atherosclerosis. J Clin Invest 107: 1227-1234.
21. Ye, S., C. R. Gale, and C. N. Martyn. 2003. Variation in the matrix metalloproteinase-1 gene and risk of coronary heart disease. European heart journal 24: 1668-1671.
22. Argraves, K. M., P. J. Gazzolo, E. M. Groh, B. A. Wilkerson, B. S. Matsuura, W. O. Twal, S. M. Hammad, and W. S. Argraves. 2008. High density lipoprotein-associated sphingosine 1-phosphate promotes endothelial bather function. J Biol Chem 283: 25074-25081.
23. Murata, N., K. Sato, J. Kon, H. Tomura, M. Yanagita, A. Kuwabara, M. Ui, and F. Okajima. 2000. Interaction of sphingosine 1-phosphate with plasma components, including lipoproteins, regulates the lipid receptor-mediated actions. Biochem J 352 Pt 3: 809-815.
24. Matsuo, Y., S. I. Miura, A. Kawamura, Y. Uehara, K. A. Rye, and K. Saku. 2006. Newly developed reconstituted high-density lipoprotein containing sphingosine-1-phosphate induces endothelial tube formation. Atherosclerosis.
25. Kuller, L. H., J. E. Eichner, T. J. Orchard, G. A. Grandits, L. McCallum, and R. P. Tracy. 1991. The relation between serum albumin levels and risk of coronary heart disease in the Multiple Risk Factor Intervention Trial. American journal of epidemiology 134: 1266-1277.
26. Deutschman, D. H., J. S. Carstens, R. L. Klepper, W. S. Smith, M. T. Page, T. R. Young, L. A. Gleason, N. Nakajima, and R. A. Sabbadini. 2003. Predicting obstructive coronary artery disease with serum sphingosine-1-phosphate. Am Heart J 146: 62-68.
27. Argraves, K. M., and W. S. Argraves. 2007. HDL serves as an S1P signaling platform mediating a multitude of cardiovascular effects. J Lipid Res 48: 2325-2333.
28. Yatomi, Y., Y. Igarashi, L. Yang, N. Hisano, R. Qi, N. Asazuma, K. Satoh, Y. Ozaki, and S. Kume. 1997. Sphingosine 1-phosphate, a bioactive sphingolipid abundantly stored in platelets, is a normal constituent of human plasma and serum. Journal of biochemistry 121: 969-973.
29. Gordon, T., W. P. Castelli, M. C. Hjortland, W. B. Kannel, and T. R. Dawber. 1977. High density lipoprotein as a protective factor against coronary heart disease. The Framingham Study. The American journal of medicine 62: 707-714.
30. Gordon, T., W. B. Kannel, W. P. Castelli, and T. R. Dawber. 1981. Lipoproteins, cardiovascular disease, and death. The Framingham study. Archives of internal medicine 141: 1128-1131.
31. Gordon, D. J., J. L. Probstfield, R. J. Garrison, J. D. Neaton, W. P. Castelli, J. D. Knoke, D. R. Jacobs, Jr., S. Bangdiwala, and H. A. Tyroler. 1989. High-density lipoprotein cholesterol and cardiovascular disease. Four prospective American studies. Circulation 79: 8-15.
32. Ansell, B. J., M. Navab, S. Hama, N. Kamranpour, G. Fonarow, G. Hough, S. Rahmani, R. Mottahedeh, R. Dave, S. T. Reddy, and A. M. Fogelman. 2003. Inflammatory/antiinflammatory properties of high-density lipoprotein distinguish patients from control subjects better than high-density lipoprotein cholesterol levels and are favorably affected by simvastatin treatment. Circulation 108: 2751-2756.
33. Pettus, B. J., A. Bielawska, B. J. Kroesen, P. D. Moeller, Z. M. Szulc, Y. A. Hannun, and M. Busman. 2003. Observation of different ceramide species from crude cellular extracts by normal-phase high-performance liquid chromatography coupled to atmospheric pressure chemical ionization mass spectrometry. Rapid Commun Mass Spectrom 17: 1203-1211.
34. Pettus, B. J., B. J. Kroesen, Z. M. Szulc, A. Bielawska, J. Bielawski, Y. A. Hannun, and M. Busman. 2004. Quantitative measurement of different ceramide species from crude cellular extracts by normal-phase high-performance liquid chromatography coupled to atmospheric pressure ionization mass spectrometry. Rapid Commun Mass Spectrom 18: 577-583.
35. Giaever, I., and C. R. Keese. 1991. Micromotion of mammalian cells measured electrically. Proc Natl Acad Sci USA 88: 7896-7900.
36. Kataoka, N., K. Iwaki, K. Hashimoto, S. Mochizuki, Y. Ogasawara, M. Sato, K. Tsujioka, and F. Kajiya. 2002. Measurements of endothelial cell-to-cell and cell-to-substrate gaps and micromechanical properties of endothelial cells during monocyte adhesion. Proc Natl Acad Sci USA 99: 15638-15643.
37. Argraves, K. M., B. A. Wilkerson, W. S. Argraves, P. A. Fleming, L. M. Obeid, and C. J. Drake. 2004. Sphingosine-1-phosphate signaling promotes critical migratory events in vasculogenesis. J Biol Chem 279: 50580-50590.
38. Schaphorst, K. L., E. Chiang, K. N. Jacobs, A. Zaiman, V. Natarajan, F. Wigley, and J. G. Garcia. 2003. Role of sphingosine-1 phosphate in the enhancement of endothelial barrier integrity by platelet-released products. Am J Physiol Lung Cell Mol Physiol 285: L258-267.
39. Xu, M., C. L. Waters, C. Hu, R. B. Wysolmerski, P. A. Vincent, and F. L. Minnear. 2007. Sphingosine 1-Phosphate Rapidly Increases Endothelial Bather Function Independently of VE-cadherin but Requires Cell Spreading and Rho Kinase. Am J Physiol Cell Physiol.
40. Peng, X., P. M. Hassoun, S. Sammani, B. J. McVerry, M. J. Burne, H. Rabb, D. Pearse, R. M. Tuder, and J. G. Garcia. 2004. Protective effects of sphingosine 1-phosphate in murine endotoxin-induced inflammatory lung injury. American journal of respiratory and critical care medicine 169: 1245-1251.
41. Finigan, J. H., S. M. Dudek, P. A. Singleton, E. T. Chiang, J. R. Jacobson, S. M. Camp, S. Q. Ye, and J. G. Garcia. 2005. Activated protein C mediates novel lung endothelial barrier enhancement: role of sphingosine 1-phosphate receptor transactivation. J Biol Chem 280: 17286-17293.
42. Garcia, J. G., F. Liu, A. D. Verin, A. Birukova, M. A. Dechert, W. T. Gerthoffer, J. R. Bamberg, and D. English. 2001. Sphingosine 1-phosphate promotes endothelial cell barrier integrity by Edg-dependent cytoskeletal rearrangement. J Clin Invest 108: 689-701.
43. Lee, M. J., M. Evans, and T. Hla. 1996. The inducible G protein-coupled receptor edg-1 signals via the G(i)/mitogen-activated protein kinase pathway. J Biol Chem 271: 11272-11279.
44. Taha, T. A., K. M. Argraves, and L. M. Obeid. 2004. Sphingosine-1-phosphate receptors: receptor specificity versus functional redundancy. Biochim Biophys Acta 1682: 48-55.
45. Vogel, C., A. Bauer, M. Wiesnet, K. T. Preissner, W. Schaper, H. H. Marti, and S. Fischer. 2007. Flt-1, but not Flk-1 mediates hyperpermeability through activation of the PI3-K/Akt pathway. Journal of cellular physiology 212: 236-243.
46. Wachtel, M., K. Frei, E. Ehler, C. Bauer, M. Gassmann, and S. M. Gloor. 2002. Extracellular signal-regulated protein kinase activation during reoxygenation is required to restore ischaemia-induced endothelial barrier failure. Biochem J 367: 873-879.
47. Lee, J. F., Q. Zeng, H. Ozaki, L. Wang, A. R. Hand, T. Hla, E. Wang, and M. J. Lee. 2006. Dual roles of tight junction-associated protein, zonula occludens-1, in sphingosine 1-phosphate-mediated endothelial chemotaxis and bather integrity. J Biol Chem 281: 29190-29200.
48. Park, K. S., M. K. Kim, H. Y. Lee, S. D. Kim, S. Y. Lee, J. M. Kim, S. H. Ryu, and Y. S. Bae. 2007. S1P stimulates chemotactic migration and invasion in OVCAR3 ovarian cancer cells. Biochem Biophys Res Commun 356: 239-244.
49. Wu, J., S. Spiegel, and T. W. Sturgill. 1995. Sphingosine 1-phosphate rapidly activates the mitogen-activated protein kinase pathway by a G protein-dependent mechanism. J Biol Chem 270: 11484-11488.
50. Lee, M. J., S. Thangada, J. H. Paik, G. P. Sapkota, N. Ancellin, S. S. Chae, M. Wu, M. Morales-Ruiz, W. C. Sessa, D. R. Alessi, and T. Hla. 2001. Akt-mediated phosphorylation of the G protein-coupled receptor EDG-1 is required for endothelial cell chemotaxis. Mol Cell 8: 693-704.
51. Osinde, M., F. Mullershausen, and K. K. Dev. 2007. Phosphorylated FTY720 stimulates ERK phosphorylation in astrocytes via S1P receptors. Neuropharmacology 52: 1210-1218.
52. Schnohr, P., G. Jensen, J. Nyboe, and A. Eybjaerg Hansen. 1977. [The Copenhagen City Heart Study. A prospective cardiovascular population study of 20,000 men and women]. Ugeskrift for laeger 139: 1921-1923.
53. Schnohr, P., J. S. Jensen, H. Scharling, and B. G. Nordestgaard. 2002. Coronary heart disease risk factors ranked by importance for the individual and community. A 21 year follow-up of 12 000 men and women from The Copenhagen City Heart Study. European heart journal 23: 620-626.
54. Bu, S., M. Yamanaka, H. Pei, A. Bielawska, J. Bielawski, Y. A. Hannun, L. Obeid, and M. Trojanowska. 2006. Dihydrosphingosine 1-phosphate stimulates MMP1 gene expression via activation of ERK1/2-Ets1 pathway in human fibroblasts. Faseb J 20: 184-186.
55. Lemaitre, V., T. K. O'Byrne, A. C. Borczuk, Y. Okada, A. R. Tall, and J. D'Armiento. 2001. ApoE knockout mice expressing human matrix metalloproteinase-1 in macrophages have less advanced atherosclerosis. J Clin Invest 107: 1227-1234.
56. Ye, S., C. R. Gale, and C. N. Martyn. 2003. Variation in the matrix metalloproteinase-1 gene and risk of coronary heart disease. European heart journal 24: 1668-1671.
57. Nakajima, K., T. Saito, A. Tamura, M. Suzuki, T. Nakano, M. Adachi, A. Tanaka, N. Tada, H. Nakamura, E. Campos, and et al. 1993. Cholesterol in remnant-like lipoproteins in human serum using monoclonal anti apo B-100 and anti apo A-I immunoaffinity mixed gels. Clinica chimica acta; international journal of clinical chemistry 223: 53-71.
58. Schlitt, A., M. R. Hojjati, H. von Gizycki, K. J. Lackner, S. Blankenberg, B. Schwaab, J. Meyer, H. J. Rupprecht, and X. C. Jiang. 2005. Serum sphingomyelin levels are related to the clearance of postprandial remnant-like particles. J Lipid Res 46: 196-200.
59. Venkataraman, K., Y. M. Lee, J. Michaud, S. Thangada, Y. Ai, H. L. Bonkovsky, N. S. Parikh, C. Habrukowich, and T. Hla. 2008. Vascular endothelium as a contributor of plasma sphingosine 1-phosphate. Circ Res 102: 669-676.
60. Paigen, B., A. Morrow, P. A. Holmes, D. Mitchell, and R. A. Williams. 1987. Quantitative assessment of atherosclerotic lesions in mice. Atherosclerosis 68: 231-240.
61. Nordestgaard, B. G., R. Wootton, and B. Lewis. 1995. Selective retention of VLDL, IDL, and LDL in the arterial intima of genetically hyperlipidemic rabbits in vivo. Molecular size as a determinant of fractional loss from the intima-inner media. Arterioscler Thromb Vasc Biol 15: 534-542.
62.

We claim:

1. A method of diagnosing cardiovascular disease in a subject, comprising the steps of:
a. collecting body fluid from the subject;
b. measuring the level of at least one sphingolipid in the body fluid;
c. diagnosing cardiovascular disease in a subject based on the measured level of sphingolipids.

2. A method of predicting cardiovascular disease in a subject, comprising the steps of:
a. collecting body fluid from the subject;
b. measuring the level of at least one sphingolipid in the body fluid;
c. predicting cardiovascular disease in a subject based on the measured level of sphingolipids.

3. A method of identifying a subject at risk of developing cardiovascular disease in a subject, comprising the steps of:
a. collecting body fluid from the subject;
b. measuring the level of at least one sphingolipid in the body fluid;
c. identifying a subject at risk of developing cardiovascular disease based on the measured level of sphingolipids.

4. The method of claim 3, wherein one or more steps is performed by a machine.

5. The method of claim 4, wherein the machine is LCMS.

6. The method of claim 3, wherein the sphingolipid is associated with HDL-C or albumin.

7. The method of claim 3, wherein the body fluid is blood or plasma.

8. The method of claim 3, wherein multiple sphingolipids are measured in the body fluid.

9. The method of claim 3, wherein the sphingolipids are S1P, DH-S1P or C24:1-ceramide.

10. The method of claim 3, wherein a subject is identified to be at risk of developing cardiovascular disease when the measured level of at least one sphingolipids is at least 10%, 15%, 20%, 25%, 30%, 35%, 40%, 45% or 50% lower than a relevant average standard level of sphingolipids in subjects without cardiovascular disease.

11. The method of claim 3, wherein a subject is identified to be at risk of developing cardiovascular disease when the measured level of at least one sphingolipid is at least 25%, 30%, 35%, 40%, 45% or 50% lower than a relevant average standard level of sphingolipids in subjects without cardiovascular disease.

12. The method of claim 3, wherein a subject is identified to be at risk of developing cardiovascular disease when the measured level of S1P is at least 0.3 μM, 0.4 μM, 0.5 μM, 0.6 μM, 0.7 μM, 0.8 μM, 0.9 μM or 1.0 μM lower than a relevant average standard level of S1P in subjects without cardiovascular disease.

13. The method of claim 3, wherein a subject is identified to be at risk of developing cardiovascular disease when the measured level of DH-S1P is at least 0.04 μM, 0.06 μM, 0.08 μM, 0.1 μM, 1.2 μM, 1.4 μM, 1.6 μM, 1.8 μM or 2.0 μM lower than a relevant average standard level of DH-S1P in subjects without cardiovascular disease.

14. The method of claim 3, wherein a subject is identified to be at risk of developing cardiovascular disease when the measured level of C24:1-ceramide is at least 0.04 μM, 0.05 μM, 0.06 μM, 0.07 μM, 0.08 μM, 0.09 μM, or 0.1 μM lower than a relevant average standard level of C24:1-ceramide in subjects without cardiovascular disease.

15. The method of claim 3, wherein a subject is identified to be at risk of developing cardiovascular disease when the [S1P] μM/[apoAI] μM ratio is at least 0.005, 0.007, 0.009, 0.011 or 0.013 lower than a relevant average standard level of [S1P] μM/[apoAI] μM ratio in subjects without cardiovascular disease.

16. The method of claim 3, wherein a subject is identified to be at risk of developing cardiovascular disease when the [DH-S1P] μM/[apoAI] μM ratio is at least 0.0005, 0.0007, 0.0009, 0.0011 or 0.0013 lower than a relevant average standard level of [DH-S1P] μM/[apoAI] μM ratio in subjects without cardiovascular disease.

17. The method of claim 3, wherein a subject is identified to be at risk of developing cardiovascular disease when the [C24:1-ceramide] μM/[apoAI] μM ratio is at least 0.0005, 0.0007, 0.0009, 0.0011 or 0.0013 lower than a relevant average standard level of [C24:1-ceramide] μM/[apoAI] μM ratio in subjects without cardiovascular disease.

18. The method of claim 3, wherein the cardiovascular disease is ischemic heart disease.

19. The method claim 3, wherein the subject does not have conventional risk factors associated with cardiovascular disease.

20. A method of treating cardiovascular disease in a subject, comprising administering a composition elevating sphingolipid levels in a subject.
